SEQUENCE LISTING (5 sequences)

SEQ ID NO: 1 - protein, 16 aa, Artificial Sequence
  Laminin polypeptide amino acid sequence
KSIKVAVRSYIGSRCV

SEQ ID NO: 2 - protein, 5 aa, Artificial Sequence
  Laminin polypeptide amino acid sequence
IKVAV

SEQ ID NO: 3 - protein, 5 aa, Artificial Sequence
  Laminin polypeptide amino acid sequence
YIGSR

SEQ ID NO: 4 - protein, 154 aa, Homo sapiens [sequence omitted]

SEQ ID NO: 5 - DNA, 981 bp, Homo sapiens
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

(19) United States
(12) Patent Application Publication
(10) Pub. No.: US 2022/0226432 A1
(43) Pub. Date: Jul. 21, 2022

(54) DEFERRED TREATMENT OF NERVE INJURIES

(71) Applicants: Ramot at Tel-Aviv University Ltd., Tel-Aviv (IL); The Medical Research, Infrastructure and Health Services Fund of the Tel Aviv Medical Center, Tel-Aviv (IL)

(72) Inventors: Zvi NEVO, Tel-Aviv (IL); Shimon ROCHKIND, Tel-Aviv (IL)

(73) Assignees: Ramot at Tel-Aviv University Ltd., Tel-Aviv (IL); The Medical Research, Infrastructure and Health Services Fund of the Tel Aviv Medical Center, Tel-Aviv (IL)

(21) Appl. No.: 17/617,043

(22) PCT Filed: Jun. 4, 2020
(86) PCT No.: PCT/IL2020/050629

§ 371 Date: Dec. 7, 2021

Related U.S. Application Data
(60) Provisional application No. 62/858,343, filed on Jun. 7, 2019.

(51) Int. Cl. A61K 38/17; A61K 31/728; A61K 9/00; A61K 31/355; A61K 38/02; A61K 38/44; A61P 25/28

(52) U.S. Cl. CPC A61K 38/1709; A61K 31/728; A61K 9/0024; A61P 25/28; A61K 38/02; A61K 38/446; A61K 31/355

(57) ABSTRACT

Deferred treatment of nerve injuries is provided. Accordingly, there is provided a method of deferred treatment of a nerve injury in a subject in need thereof, the method comprising implanting at least 1 week following onset or diagnosis of the nerve injury in the subject a composition comprising a hyaluronic acid, a laminin polypeptide and an antioxidant at or near the nerve injury of the subject.

### RELATED APPLICATION

This application claims priority from U.S. Patent Application No. 62/858,343 filed on Jun. 7, 2019, the contents of which are incorporated herein by reference in their entirety.

### SEQUENCE LISTING STATEMENT

The ASCII file, entitled 82609 SequenceListing.txt, created on 4 Jun. 2020, comprising 3,799 bytes, submitted concurrently with the filing of this application is incorporated herein by reference.

### FIELD AND BACKGROUND OF THE INVENTION

The present invention, in some embodiments thereof, relates to deferred treatment of nerve injuries.

Nerve and brain injuries, including traumatic and degenerative injuries to peripheral nerves and/or the spinal cord (SCI) have no successful treatment to date. With respect to SCI for example, even a mild contusion to the spinal cord can result in massive neuronal and glial cell loss, demyelination, cavitation, and glial scarring. Pathological changes such as these have detrimental functional effects causing loss of sensory perception, distal motor paralysis, and severe functional impairment, with the final outcome depending upon axonal sparing, remyelination, and possibly neural regeneration. Similar effects are also observed with many neurodegenerative disorders including, inter alia, Alzheimer's Disease, Parkinson's Disease, Multiple Sclerosis, Amyotriphic Lateral Sclerosis, multiple-system degenerations, cerebellar degeneration, and the like.

Recent advances have identified compounds that protect neuronal elements immediately following injury and/or stimulate the growth of axons when administered within several days of the injury (i.e. during the acute after-injury period) (e.g. S. Rossignol et al, J. Neurosci. 27:11782-92 (2007); B. P. Liu et al, Philos. Trans. R. Soc. Lond. B. Biol ScL 3(57:1593-1610 (2006); S. Li et al, J. Neurosci. 24:10511-20 (2004); J. K. Lee et al, J. Neurosci. 24:6209-17 (2004)]. However, most of the pharmacological agents identified to date for nerve injuries have been considered incapable of re-activating axonal growth and recovery in the much more prevalent condition of chronic nerve injury.

Hydrated gels (hydrogels) are viscous, semisolid entities at physiological temperatures and pH which can be used for tissue engineering and regenerative medicine. For example, hyaluronic acid-based hydrogels provide a growth supportive milieu for cells and tissues such as for nerve regeneration (Suzuki et. al., 2003; Itoh et. al., 2005), while guiding migration and regeneration of nutritional-trophic and anti-oxidative agents.

International Patent Application Publication No. WO2009/022339 discloses the use of a hyaluronic acid-based hydrogel containing the antioxidant sodium dismutase and a laminin peptide for neural tissue regeneration and repair.

### SUMMARY OF THE INVENTION

According to an aspect of some embodiments of the present invention there is provided a method of deferred treatment of a nerve injury in a subject in need thereof, the method comprising implanting at least 1 week following onset or diagnosis of the nerve injury in the subject a composition comprising a hyaluronic acid, a laminin polypeptide and an antioxidant at or near the nerve injury of the subject, thereby treating the nerve injury in the subject.

According to some embodiments of the invention, the implanting is effected at least two weeks following onset or diagnosis of the nerve injury.

According to some embodiments of the invention, the implanting is effected within 3 years following onset or diagnosis of the nerve injury.

According to an aspect of some embodiments of the present invention there is provided a composition comprising a hyaluronic acid, a laminin polypeptide and an antioxidant for use in deferred treatment of a nerve injury in a subject in need thereof, wherein the deferred treatment is at least 1 week following onset or diagnosis of the nerve injury in the subject.

According to an aspect of some embodiments of the present invention there is provided a kit comprising a composition comprising a hyaluronic acid, a laminin polypeptide and an antioxidant; and instructions for use in deferred treatment of a nerve injury in a subject in need thereof, wherein the deferred treatment is at least 1 week following onset or diagnosis of the nerve injury in the subject.

According to some embodiments of the invention, the deferred treatment is at least two weeks following onset or diagnosis of the nerve injury.

According to some embodiments of the invention, the deferred treatment is within 3 years following onset or diagnosis of the nerve injury.

According to some embodiments of the invention, the nerve injury is caused by a trauma and not by a disease.

According to some embodiments of the invention, the nerve injury is a chronic nerve injury.

According to some embodiments of the invention, the nerve injury is part of the peripheral nervous system (PNS).

According to some embodiments of the invention, the nerve injury is part of the central nervous system (CNS).

According to some embodiments of the invention, the nerve injury comprises spinal cord injury (SCI).

According to some embodiments of the invention, the nerve injury comprises traumatic brain injuries (TBI) or traumatic optic neuropathy (TON).

According to some embodiments of the invention, the antioxidant is vitamin E.

According to some embodiments of the invention, the antioxidant is superoxide dismutase (SOD).

According to some embodiments of the invention, the SOD comprises the amino acid sequence set forth by SEQ ID NO: 4.

According to some embodiments of the invention, the laminin polypeptide is set forth by SEQ ID NO: 1.

According to some embodiments of the invention, the antioxidant is vitamin E and the laminin polypeptide is set forth by SEQ ID NO: 1.

According to some embodiments of the invention, the antioxidant is superoxide dismutase (SOD) comprising the amino acid sequence set forth by SEQ ID NO: 4 and the laminin polypeptide is set forth by SEQ ID NO: 1.

According to some embodiments of the invention, the hyaluronic acid, the antioxidant and the laminin polypeptide are cross linked.

According to some embodiments of the invention, the composition further comprises cells.

According to some embodiments of the invention, the cells are stem cells.

According to some embodiments of the invention, the stem cells differentiate into neuronal cells when seeded in the composition.

According to some embodiments of the invention, the composition further comprises an anti-gliotic agent and/or a neuronal supporting agent.

According to some embodiments of the invention, the anti-gliotic agent and/or the neuronal supporting agent is selected from the group consisting of chondroitinase ABC, anti Nogo A, Copolymer 1, serotonin, a TNFα inhibitor and/or an IL-1 inhibitor.

According to some embodiments of the invention, the antioxidant is vitamin E, the laminin polypeptide is set forth by SEQ ID NO: 1 and the agent is Copolymer 1.

According to some embodiments of the invention, the antioxidant is superoxide dismutase (SOD) comprising the amino acid sequence set forth by SEQ ID NO: 4, the laminin polypeptide is set forth by SEQ ID NO: 1 and the agent is Copolymer 1.

According to some embodiments of the invention, the composition is formulated for local administration.

According to some embodiments of the invention, the composition is formulated as a hydrogel.

According to some embodiments of the invention, the composition is formulated as a matrix.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 demonstrate the experimental design of the rabbit chronic peripheral nerve injury model.

FIG. 2 is a graph demonstrating mean body weight of rabbits in the chronic peripheral injury model.

FIG. 3 is a graph demonstrating left limb (injured) CAMP normalized amplitude. The amplitude values of the CAMPs measured at weeks 7, 11, 15 and 23 weeks after treatment, were normalized to the amplitude measured 5 weeks prior the treatment surgery (Surgery 2). Results are presented as mean±SEM. Asterisk represents statistical significance: * p<0.05 vs. NeuraGen® Nerve Guide, using one-way ANOVA followed by Dunnett's test; #p<0.05 vs. week 7, using one-way ANOVA followed by Tukey HSD test.

FIG. 4 demonstrates MBP staining assessing regeneration. The graph displays the mean relative area (mean±SEM) of MBP in Proximal and Distal sections. Asterisk represents statistical significance: * p<0.1 using two-tailed Student's T-test vs. NeuraGen® Nerve Guide; ** p<0.01 using two-tailed Student's T-test vs. proximal section; #p<0.05 using two-tailed Student's T-test vs. proximal section; †p<0.1 using one-tailed Student's T-test vs. proximal section.

FIG. 5 shows representative histological images demonstrating MBP staining. The proximal and distal sections of the tibial portion of the sciatic nerve of each treatment are displayed. Images were taken at magnification of ×20.

FIG. 6 is a graph demonstrating mean body weight of rats in the chronic spinal cord injury model.

FIG. 7 is a graph demonstrating the effect of AGRG on BBB Locomotor rating scale in the rat chronic spinal cord injury model. The graph displays the BBB score of 1 week prior to the 2^{nd} surgery (i.e., 4 weeks following the 1^{St} surgery), at week 0 (following the 2^{nd} surgery) and at the end of the study (week 22/24).

FIGS. 8A-B demonstrate MBP staining assessing regeneration. FIG. 8A is a graph displaying the mean±SE of MBP in each section (CRAN, MID, CAUD). FIG. 8B is a graph demonstrating the mean±SE of MBP only in the MID section. *p<0.01 using one-way ANOVA followed by Tukey's multiple comparisons vs. untreated.

FIG. 9 shows representative histological images demonstrating MBP staining. The CRAN, MID and CAUD sections of the spinal cord of each treatment are displayed. Images were taken at magnification of ×20.

### DESCRIPTION OF SPECIFIC EMBODIMENTS OF THE INVENTION

The present invention, in some embodiments thereof, relates to deferred treatment of nerve injuries.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

While reducing specific embodiments of the invention to practice, the present inventors have now uncovered that a composition comprising hyaluronic acid, an anti-oxidant, a laminin peptide (SEQ ID NO: 1) and optionally an anti-gliotic agent (e.g. Copolymer 1) can be used for deferred treatment of nerve injuries. When formulated as a gel the hydrogel is referred to herein as GRG for guiding regenerative gel or AGRG for anti-gliotic guiding regenerative gel.

As is illustrated hereinunder and in the Examples section, which follows, the present inventors demonstrate that implanting GRG or AGRG had a regenerative effect in a chronic peripheral nerve injury rabbit model and a chronic spinal nerve injury rat model (Examples 1 and 2 in the Examples section which follows).

Hence, according to an aspect of the present invention there is provided a method of deferred treatment of a nerve injury in a subject in need thereof, the method comprising implanting at least 1 week following onset or diagnosis of the nerve injury in the subject a composition comprising a hyaluronic acid, a laminin polypeptide and an antioxidant at or near the nerve injury of the subject, thereby treating the nerve injury in the subject.

According to an additional or an alternative aspect of the present invention, there is provided a composition comprising a hyaluronic acid, a laminin polypeptide and an antioxidant for use in deferred treatment of a nerve injury in a subject in need thereof, wherein said deferred treatment is at least 1 week following onset or diagnosis of the nerve injury in the subject.

As used herein, the term “subject” refers to a mammalian subject (e.g., human being) of any gender and any age including neonatal, infant, juvenile, adolescent, adult and elderly adult which suffer from the pathology (i.e. nerve injury) as described below.

Veterinary uses are also contemplated. Thus, according to specific embodiments, the components of some embodiments of the present invention (e.g. of the composition, the matrix, the hydrogel) are selected avoiding xeno responses.

The terms “treatment” and “treating”, which are interchangeably used herein, refer to inhibiting or arresting the development of a pathology (e.g. nerve injury) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology. According to specific embodiments, treating comprises increasing survival, increasing motor function, increasing sensory function and/or decreasing pain.

As used herein, the phrase “deferred treatment” refers to treating a subject at least 1 weeks following onset or diagnosis of a nerve injury.

According to specific embodiments, the deferred treatment is at least 2 weeks, at least 3 weeks, at least 4 weeks, at least 2 months, at least 6 months, at least 12 months, at least 2 years following onset or diagnosis of the nerve injury, each possibility represents a separate embodiment of the present invention.

As used herein the phrase “nerve injury” refers to any disorder, disease, or condition exhibiting damage (i.e., non-functioning tissue, cancerous or pre-cancerous tissue, broken tissue, fractured tissue, fibrotic tissue, or ischemic tissue) or loss (e.g., following a trauma, an infectious disease, a genetic disease, and the like) of neuronal tissue which requires tissue regeneration.

According to specific embodiments the nerve injury is caused by trauma and not by a disease.

According to specific embodiments the nerve injury is a chronic nerve injury.

According to specific embodiments the neuronal tissue and/or the nerve injury is part of the peripheral nervous system (PNS).

According to specific embodiments the neuronal tissue and/or the nerve injury is part of the central nervous system (CNS).

The term “central nervous system (CNS)”, as used herein can refer to a subject's brain, spinal cord and/or optic nerve.

According to specific embodiments, the nerve injury comprises spinal cord injury.

As used herein, the phrase “spinal cord injury (SCI)” refers to an injury to the spinal cord that is caused by trauma and not by disease. Spinal cord injuries have many causes; according to specific embodiments, the SCI is caused by a major trauma from motor vehicle accidents, falls, sports injuries or violence. Depending on where the spinal cord and nerve roots are damaged, the symptoms can vary widely e.g. from pain to paralysis to incontinence. The SCIs can be incomplete or complete injury which means a total loss of function. According to specific embodiments, the SCI is complete SCI.

According to specific embodiments, the nerve injury comprises traumatic brain injury (TBI).

As used herein, the phrase “traumatic brain injury (TBI)” refers to brain injury caused by trauma and not by disease. TBIs have many causes; according to specific embodiment, the TBI is caused by falls, vehicle collisions, sports collisions or combats. The phrase includes both mild and severe TBI including closed-head injuries, concussions or contusions and penetrating head injuries.

According to specific embodiments, the nerve injury comprises traumatic optic neuropathy (TON).

As used herein, the phrase “traumatic optic neuropathy (TON)” refers to injury to the optic nerve caused by trauma and not by disease. According to specific embodiments, TON results in vision loss, which may be partial or complete. TONs have many causes; according to specific embodiments, the TON is caused by an anatomical disruption of the optic nerve fibers from penetrating orbital trauma, bone fragments within the optic canal or nerve sheath hematomas.

As noted, the compositions of some embodiments of the present invention comprise a hyaluronic acid, a laminin polypeptide and an antioxidant.

As used herein, the term “hyaluronic acid (HA)”, also known as hyaluronan, hyaluronate, refers to an unsulphated glycosaminoglycan composed of repeating disaccharide units of N-acetylglucosamine (GlcNAc) and glucuronic acid (GlcUA) linked together by alternating beta-1, 4 and beta-1, 3 glycosidic bonds. According to specific embodiments the hyaluronic acid is Na-HA. According to specific embodiments, the hyaluronic acid has a molecular weight from about 10⁴ Daltons to about 3×10⁶ Daltons. The molecular weight of HA can be evaluated by e.g. viscosity measurement with a digital viscosimeter Brookfield brand Cone/Plate DVII+Per (Brookfield Engineering Laboratories Inc. Middleboro, Mass. 02346-1031 USA). The molecular weight of HA can be calculated as well by the discrepancy between the figure obtained in Dische's assays versus the data obtained by Park-Johnson (Park J. T. Johnson M. J. A submicrodetermination of glucose J. Biol. Chem. 181, 149-151, 1949) determination for reducing sugars.

The hyaluronic acid described herein includes naturally occurring HA synthetic HA or a combination of same. According to specific embodiments, the hyaluronic acid can be extracted and isolated from an organism such as rooster combs or umbilical cords or from bacterial cultures such as those of hemolytic group A or C Streptococci, or can be synthetically produced using methods which are well known in the art.

According to specific embodiments of the invention, the hyaluronic acid is pure enough from chemical or biological constituents so that it is biologically inert having a low rate of reactivity with other substances under ordinary conditions.

According to specific embodiments of the invention, the hyaluronic acid is pure enough so that it is biocompatible, e.g., when in contact with cells, tissues or body fluid of an organism does not induce adverse effects such as immunological reactions and/or rejections, cellular death, and the like.

According to specific embodiments, the hyaluronic acid is at least 80%, at least 90%, at least 95%, at least 98% or at least 99% pure.

According to specific embodiments, the hyaluronic acid is analytical (i.e. 99.5%-100%) or pharmaceutical grade (98%-100%) hyaluronic acid.

According to specific embodiments, the hyaluronic acid is a hyaluronic acid such as commercially obtained from Lifecore Biomedical LLC Cat No. HAHA15M-1.

The hyaluronic acid described herein is capable of forming highly hydrated gels in aqueous solutions.

Total content of HA in the composition can be determined by methods known in the art such as, but not limited to the content of uronic acids (lucuronic acid) by the routine test of Dische (Dische Z. A new specific color reaction of hexuronic Acids. J. Biol. Chem, 167, 189-197, 1947) employing the carbazol reagent.

As used herein the term “laminin” refers to the family of extracellular matrix glycoproteins, which form the major noncollagenous constituent of basement membrane.

Laminins have been implicated in a wide variety of biological processes including cell adhesion, differentiation, migration, signaling, neurite outgrowth and metastasis. Laminins are composed of 3 non identical chains:laminin alpha, beta and gamma, each encoded by a distinct gene.

As used herein the phrase “laminin polypeptide” refers to an amino acid sequence which comprises at least 4 consecutive amino acids of a laminin polypeptide and which exhibits a biological activity (e.g., support cell survival, growth, proliferation, differentiation and/or migration).

According to some embodiments of the invention the laminin polypeptide can include an amino acid sequence of a laminin alpha-chain such as LAMA1 (e.g., GenBank Accession No. NP_005550.2), LAMA2 (e.g., GenBank Accession Nos. NP_000417.2 and NP_001073291.1), LAMA3 (e.g., GenBank Accession Nos. NP_937762.1 and NP_000218.2), LAMA4 (e.g., GenBank Accession Nos. NP_001098677.1, NP_001098676.1, NP_002281.2, NP_001098679.1, and NP_001098678.1), and LAMA5 (e.g., GenBank Accession No. NP_005551.3); a laminin beta-chain such as LAMB1 (e.g., GenBank Accession No. NP_002282.1), LAMB2 (e.g., GenBank Accession No. NP_002283.3), LAMB3 (e.g., GenBank Accession Nos. NP_000219.2 and NP_001017402.1) and LAMB4 (e.g., GenBank Accession No. NP_031382.2); and/or a laminin gamma-chain such as LAMC1 (e.g., GenBank Accession No. NP_002284.3), LAMC2 (e.g., GenBank Accession Nos. NP_005553.2 and NP_061486.2) and LAMC3 (e.g., GenBank Accession No. NP_006050.3).

According to specific embodiments of the invention, the laminin polypeptide includes a repeated amino acid sequence (e.g., a 4 or 5 amino acid repeated sequence) of a laminin sequence.

Non-limiting examples of laminin polypeptides which can be included in the composition of some embodiments of the invention include the peptides set forth in SEQ ID NOs: 1, 2 or 3. [KSIKVAVRSYIGSRCV (SEQ ID NO: 1), IKVAV (SEQ ID NO: 2), YIGSR (SEQ ID NO: 3)].

According to specific embodiments, the laminin polypeptide is set forth by SEQ ID NO: 1 (KSIKVAVRSYIGSRCV).

According to specific embodiments the laminin polypeptide is 5-50, 5-25, 5-20, 16-50 or 16-25 amino acids long.

According to specific embodiments, the laminin polypeptide is at least 16 amino acids long but shorter than 50 amino acids long.

The terms “polypeptide” or “peptide” which are interchangeably used herein, encompass native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (˜CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of some embodiments of the present invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (e.g., synthetic, Table 2) which can be used with the present invention.

TABLE 1

| | Three-Letter | One-letter |
|---|---|---|
| Amino Acid | Abbreviation | Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino | Xaa | X |
| acid as above | | |

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N-methylhomophenylalanine | Nmhphe |
| | Nnbhm | | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The peptides of some embodiments of the present invention are preferably utilized in a linear form, although it will be appreciated that in cases where cyclicization does not severely interfere with peptide characteristics, cyclic forms of the peptide can also be utilized.

Since the present peptides can be utilized in therapeutics or diagnostics which require the peptides to be in soluble form, the peptides of some embodiments of the present invention preferably include one or more non-natural or natural polar amino acids, including but not limited to serine and threonine which are capable of increasing peptide solubility due to their hydroxyl-containing side chain.

The peptides of some embodiments of the present invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the present invention involves solid phase peptide synthesis.

Large scale peptide synthesis is described by Andersson Biopolymers 2000; 55(3):227-50.

In cases where large amounts or long polypeptides (e.g., longer than 20 amino acids) are desired, the polypeptides of some embodiments of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680, Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The composition further comprises an antioxidant which can protect cells or macromolecules (e.g., the polysaccharide) from oxidative stress (oxidative damage caused by free radicals). Thus, the antioxidant can extend the survival of the macromolecules by preventing their oxidative depolymerization.

Non-limiting examples of suitable antioxidants which can be used with specific embodiments of the present invention include molecules such as glutathione, vitamin C (sodium ascorbate), vitamin E, melatonin, coenzyme Q10, Acetyl-DL-Carnitine and N-Acetyl-D-Glucosamine, retinoic acid, L-ascorbic acid, L-selenomethionine, curcumin, N—Ac-L-cysteine, hydroquinone, glutamate, or enzymes such as catalase, superoxide dismutase, glutathione peroxidase or other peroxidases, glucose-6-phosphate dehydrogenase (G6PD) (see Osmen I., Naziroglu M., Okutan R. Comparative study of antioxidant enzymes in tissues surrounding implant in rabbits. Cell. Biochem. Funct. 24:275-281, 2006).

According to specific embodiments, the antioxidant is superoxide dismutase (SOD), vitamin E, melatonin, Coenzyme Q10, Acetyl-DL-Carnitine and/or N-Acetyl-D-Glucosamine.

According to specific embodiments, the antioxidant is vitamin E, melatonin, Coenzyme Q10, Acetyl-DL-Carnitine and/or N-Acetyl-D-Glucosamine.

According to specific embodiments, the antioxidant is vitamin E.

According to specific embodiments, the antioxidant is vitamin E and the laminin polypeptide is set forth by SEQ ID NO: 1.

As used herein, the term “vitamin E”, also referred to herein as “Tocopherol”, includes alpha, beta, gamma, and delta-tocopherols and their derivatives, conjugates, metabolites and salts. The vitamin E may also be a combination of alpha, beta, gamma, and delta-tocopherols.

According to specific embodiments, the vitamin E is alpha Tocopherol.

The vitamin E described herein includes naturally occurring vitamin E synthetic vitamin or a combination of same.

The alpha-form occurs naturally as the d-isomer known as d-alpha-tocopherol (d-2,5,7,8-tetramethyl-2-(4′,8′,12′-trimethyltridecyl)-6-chromanol). Other non-limiting forms of vitamin E which can be used with specific embodiments of the invention include: d-alpha.-tocopheryl acetate, d-alpha-tocopheryl succinate, d-alpha.-tocopheryl nicotinate and d-alpha.-tocopheryl linoleate. Also the corresponding dl forms may be used which include: dl-.alpha.-tocopherol, dl-.alpha.-tocopheryl acetate, dl-alpha-tocopheryl succinate, dl-alpha-tocopheryl nicotinate and dl-alpha.-tocopheryl linoleate and their derivatives, conjugates, metabolites and salts.

Methods of extracting, purifying or synthesizing vitamin E are known in the art. For example, naturally sourced d-alpha-tocopherol can be extracted and purified from seed oils; gamma-tocopherol can be extracted, purified, and methylated to create d-alpha-tocopherol. dl-alpha-tocopherol can be synthesized e.g. from a mixture of toluene and 2,3,5-trimethyl-hydroquinone that reacts with isophytol to all-rac-alpha-tocopherol, using iron in the presence of hydrogen chloride gas as catalyst.

According to specific embodiments, the antioxidant is superoxide dismutase (SOD).

As used herein, the term “superoxide dismutase (SOD)” E.C. No: 1.15.1.1 refers to an enzyme that alternately catalyzes the dismutation (or partitioning) of the superoxide (O₂⁻) radical into either ordinary molecular oxygen (O₂) or hydrogen peroxide (H₂O₂). Superoxide dismutase, in addition to its known activity as an antioxidant, can also serve as an anti-inflammatory agent when used in vivo. Non-limiting examples of superoxide dismutase (SOD) enzymes which can be used in the composition of some embodiments of the invention include, SOD-1 (soluble), SOD-2 (mitochondrial) or SOD-3 (extracellular), such as Homo sapiens soluble superoxide dismutase 1 (SOD-1) GenBank Accession No. NP_000445 (SEQ ID NO: 4); Homo sapiens mitochondrial superoxide dismutase 2 (SOD-2) GenBank Accession Nos. NP_001019637.1 (isoform B), NP_001019636.1 (isoform A), NP_000627.2 (isoform A); Homo sapiens extracellular superoxide dismutase 3 (SOD-3) GenBank Accession No. NP_003093.2; Saccharomyces cerevisiae SOD-1 GenBank Accession No. NP_012638.1; and Rattus norvegicus SOD-1 GenBank Accession No. NP_058746.

According to specific embodiments, the SOD comprises the amino acid sequence set forth by SEQ ID NO: 4.

According to specific embodiments, antioxidant is superoxide dismutase (SOD) comprising the amino acid sequence set forth by SEQ ID NO: 4 and the laminin polypeptide is set forth by SEQ ID NO: 1.

The antioxidant of some embodiments of the invention can be produced by recombinant techniques, e.g. as described in Hartman J R., et al., 1986 (Proc. Natl. Acad. Sci. USA, Vol: 83, pp 7142-7146). For example, a polynucleotide encoding superoxide dismutase 1 (GenBank Accession No. NM_000454; SEQ ID NO: 5) can be ligated into a nucleic acid construct suitable for expression in a host cell (e.g., bacterial cell, yeast cell, mammalian cell). Such a nucleic acid construct includes a promoter sequence for directing transcription of the polynucleotide sequence in the cell in a constitutive or inducible manner, and may also include sequences which render this vector suitable for replication and integration in prokaryotes, eukaryotes, or preferably both (e.g., shuttle vectors); transcription and translation initiation sequence, enhancers, transcription and translation terminator, and a polyadenylation signal which may increase the efficiency of mRNA translation; a signal sequence for secretion; sequences engineered to enhance stability, production, purification, yield or toxicity of the expressed polypeptide.

The antioxidant can be recovered and purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromate-focusing and differential solubilization.

According to specific embodiments of the invention, the antioxidant is provided such that it is pure enough from chemical or biological constituents to allow for the effective use of the compound or the recombinant polypeptide as an antioxidant. Activity of the anti-oxidant (e.g. SOD, vitamin E) may be determined by methods well known in the art and include measurement at 560 nm as the rate of suppression of reduction of nitrotetrazolium blue when superoxide anion radical was generated during oxidation of xanthine by xanthine oxidase.

According to specific embodiments, the anti-oxidant (e.g. SOD, vitamin E) is at least 80%, at least 90%, at least 95%, at least 98% or at least 99% pure.

According to specific embodiments, the anti-oxidant (e.g. SOD, vitamin E) is analytical or pharmaceutical grade anti-oxidant.

According to specific embodiments, the compositions described herein may further comprise other agents capable of supporting regeneration of neuronal tissue such as, but not limited to anti-gliotic agents and/or neuronal supporting agents.

According to specific embodiments, the composition described herein may further comprise an anti-gliotic agent.

As used herein, the term “gliosis” refers to a nonspecific change of glial cells e.g. astrocytes and macrophages, in response to damage to the central nervous system (CNS). Typically, gliosis involves proliferation of glial cell, hypertrophy of glial cells and secretion of connective tissue matrix substances such as proteoglycans (PGs), collagens and myelin-derived residues. Gliosis, in its extreme form, leads to the formation of a scar tissue in the CNS comprising dense fibrous network of glial cell in areas of damage resulting in inhibition of axons sprouting and restricting neuronal regeneration.

Methods of determining gliosis are known in the art and are further described in the Examples section which follows and include in-vitro methods determining neuronal cells survival and astrocytes survival and quality, biosynthesis and accumulation of inhibitory intracellular, pericellular and extracellular (ECM) components such as GAGs; and in-vivo methods determining neuronal regeneration in response to CNS injury e.g. SCI.

As used herein, the term “anti-gliotic agent” refers to an agent capable of decreasing the extent of gliosis. Typically, an anti-gliotic agent decreases the extent of gliosis by degrading the scar barrier and/or inhibiting its further formation. According to specific embodiments the decrease is at least 1.05 fold, at least 1.1 fold, at least 1.2 fold, at least 1.3 fold, at least 1.4 fold, 1.5 fold, at least 2 fold, at least 3 fold, at least 5 fold, at least 10 fold, or at least 20 fold as compared to same in the absence of the anti-gliotic agent.

According to other specific embodiments the decrease is by at least 5%, by at least a 10%, at least 20%, at least 30%, at least 40% or at least 50% as compared to same in the absence of the anti-gliotic agent.

Non limiting examples of anti-gliotic agents which can be used with specific embodiments of the present invention include Chondroitinase ABC (E.C. No 4.2.2.4), 3-D-xyloside (E.C. No 217.897.1), Collagenase Type I (E.C. No 232-582-9), Mitomycin-C(CAS No 50-07-7), MMP-3-Matrix Metalloproteinase (E.C. No 3.4.24, anti Nogo A, anti-TGFβ 1, 2 & 3, angiotensin Converting Enzyme (ACEa, E.C No 3.4.15.1), anti NG-2-domain, Decorin (e.g. human Decorin such as Uniprot accession No. P07585, PAPN-beta aminopropionyl, Mannose-6-phosphate (CAS No 3672-15-9), Oxidized recombinant human galectin-1, Copaxone (glatiamer acetate) and Tri peptide (ser-gly-gly).

According to specific embodiments, the compositions described herein may further comprise a neuronal supporting agent.

As used herein the term “neuronal supporting agent” refers to an agent capable of increasing sprouting, growth, survival and/or function of a neuronal cell. Methods of determining neuronal sprouting, growth, survival and/or function are known in the art and are further described in the Examples section which follows and include in-vitro methods determining neuronal cells growth and survival; and in-vivo methods determining neuronal regeneration in response to CNS injury e.g. SCI.

According to specific embodiments the increase is at least 1.05 fold, at least 1.1 fold, at least 1.2 fold, at least 1.3 fold, at least 1.4 fold, 1.5 fold, at least 2 fold, at least 3 fold, at least 5 fold, at least 10 fold, or at least 20 fold as compared to same in the absence of the neuronal supporting agent.

According to other specific embodiments the increase is by at least 2%, at least 5%, at least a 10%, at least 20%, at least 30%, at least 40% or at least 50% as compared to same in the absence of the neuronal supporting agent.

Non limiting examples of neuronal supporting agents which can be used with specific embodiments of the present invention include Copolymer 1 (e.g. glatiramer acetate or its trade name Copaxone®), ibuprofen, indomethacin, methylprednisolone, N-acetyl-cysteine, serotonin, a calcium channel blocker (e.g. Verapamil) a TNFα inhibitor (e.g. Etanercept, Infliximab), an IL-1 inhibitor (e.g. canacinumab, Anakinra), an agent capable of upregulating activity of peroxisome proliferator-activated receptor (PPAR, e.g. Rosiglitazone) binding agent and an agent capable of upregulating activity of SiP receptor (e.g. fingolimod).

According to specific embodiments, the neuronal supporting agent and/or the anti-gliotic agent is chondroitinase ABC, anti Nogo A, Copolymer 1, serotonin, a TNFα inhibitor and/or an IL-1 inhibitor.

According to specific embodiments, the antioxidant is vitamin E, the laminin polypeptide is set forth by SEQ ID NO: 1 and the agent is Copolymer 1 According to specific embodiments, the antioxidant is superoxide dismutase (SOD) comprising the amino acid sequence set forth by SEQ ID NO: 4, the laminin polypeptide is set forth by SEQ ID NO: 1 and the agent is Copolymer 1.

According to specific embodiments, the components of the composition are cross-linked.

According to specific embodiments, the hyaluronic acid, the antioxidant and the laminin polypeptide are cross linked.

According to specific embodiments, the hyaluronic acid, laminin polypeptide, the antioxidant and the neuronal supporting agent and/or an anti-gliotic agent are cross linked.

Cross-linking (i.e., binding via covalent or ionic bonds) of the components comprised in the composition can be performed using any cross-linking or coupling agent known in the art. Basically the principles of cross linking is combining free primary amino groups with carboxyl groups, or oxidizing in between close hydroxyl groups, forming reactive aldehydes, to interact either among themselves or with amines of additional conjugate may be formed via tiol residues.

According to specific embodiments, cross-linking does not affect the biological activities of the bonded elements.

Non-limiting examples of suitable cross-linking agents include dimethyl suberimidate (an imidoester cross linker); Bis(Sulfosuccinimidyl) suberate (BS3; an NHS-ester cross linker); formaldehyde; 1-Ethyl-3-[3-dimethylaminopropyl]carbodiimide hydrochloride (EDC; the carbodiimide cross linker); N-hydroxyuccinimide (NHS) [Mao J. S, et al., Biomaterials. 24, 1621-1629, 2003; Choi Y. S., et al., J. Biomed. Mater. Res. 48,631-639, 1999; Richert L., et al., Biomacromolecules, 5, 284-294, 2004)]; Divinyl sulfone (DVS); and genipin [Sung H. W., et al., J Biomed. Mater. Res. A, 64A:427-438, 2003; Chen S C., et al., J. Control Release. 96, 285-300, 2004; Mwale F., et al., Tissue Eng., 11, 130-40, 2005; Chen H., et al., Biomacromolecules, 7, 2091-2098, 2006]. For ex vivo or in vivo cross-linking photo-reactive amino acid analogs (e.g., diazirine analogs to leucine and methionine) can be added to the composition and following exposure to ultraviolet light, the diazirines are activated and bind to interacting side chains (e.g., carboxyl or amino groups).

According to specific embodiments of the invention, cross-linking is performed using a non-toxic and/or biocompatible agent. Examples include, but are not limited to 3-dimenthy-aminoprophyl)-N-ethyl carbodiimide (EDC-N; Sigma-Aldrich-Fluka, St Louis, Mo. 63178, Catalogue No. 03459), divinyl sulfone (DVS; Sigma, Catalogue No. V-370-0) and genipin (Sigma Catalogue No. G-4796).

According to specific embodiments, the composition described herein has combined improved activity on neural cells survival, neuronal regeneration and/or prevention of glial scar tissue growth. As used herein the phrase “combined improved activity” refers to at least additive but preferably synergistically improved activity.

It should be noted that since the components comprised in the composition of some embodiments of the invention can be prepared using synthetic or recombinant techniques they are obtainable sterile preparations of analytical or pharmaceutical grade.

According to specific embodiments of the invention, the composition is formulated as a hydrogel.

As used herein, the term “hydrogel” refers to a material comprising the composition of some embodiment of the invention and water, in which the water constitutes more than 40%.

According to specific embodiments of the invention, the hydrogel comprises at least about 50%, at least about 60% water, at least about 70% water, at least about 80% water, at least about 90% water, at least about 95% water, at least about 96% water, at least about 97% water, at least about 98% water, at least about 99% water.

Methods of generating a hydrogel are known in the art. Thus, according to specific embodiments, suspending the composition described herein in water is effected according to the teachings of International Patent Application Publication No. WO2009/022339, the contents of which are incorporated herein in their entirety.

According to specific embodiments, the hydrogel is viscous (e.g. approximately OcP during no movement and 110-130 cP during movement).

According to specific embodiments, the hydrogel is transparent.

According to specific embodiments, the hyaluronic acid is provided at a concentration range of about 0.2-1%, about 0.4-1%, about 0.7-1%, about 0.3-2%, e.g., about 0.4-1.8%, e.g., about 0.5-1.6, e.g., about 0.5-1.5%, e.g., about 0.6-1.4%, e.g., about 0.7-1.4%, about 0.8-1.2%, e.g., about 1.2% in the composition e.g. hydrogel.

According to a specific embodiment, the hyaluronic acid is provided at a concentration range of about 0.5-1.5% in the composition e.g. hydrogel.

According to a specific embodiment, the hyaluronic acid is provided at a concentration range of about 0.2-1% in the composition e.g. hydrogel.

According to a specific embodiment, the hyaluronic acid is provided at a concentration of about 0.2% in the composition e.g. hydrogel.

According to a specific embodiment, the hyaluronic acid is provided at a concentration of about 0.4% in the composition e.g. hydrogel.

According to a specific embodiment, the hyaluronic acid is provided at a concentration of about 0.7% in the composition e.g. hydrogel.

According to a specific embodiment, the hyaluronic acid is provided at a concentration of 0.2% in the composition e.g. hydrogel.

According to a specific embodiment, the hyaluronic acid is provided at a concentration of 0.4% in the composition e.g. hydrogel.

According to a specific embodiment, the hyaluronic acid is provided at a concentration of 0.7% in the composition e.g. hydrogel.

According to some embodiments, the laminin polypeptide (e.g., SEQ ID NO: 1) is provided at a concentration range of about 10-200 μg/ml, e.g., about 10-100 μg/ml e.g., about 20-100 μg/ml, e.g., about 50 μg/ml in the composition e.g. hydrogel.

According to a specific embodiment, the laminin polypeptide is provided at a concentration range of about 20-100 μg/ml in the composition e.g. hydrogel.

According to a specific embodiment, the laminin polypeptide is provided at a concentration range of about 10-100 μg/ml in the composition e.g. hydrogel.

According to some embodiments of the invention, the antioxidant is provided at a concentration range of 8 μM (e.g. for SOD about 0.25 microgram/ml) to 8 mM (e.g. for SOD about 250 microgram/ml) in the hydrogel. For example, the antioxidant can be provided at a concentration range of 0.5 μg/ml to 200 μg/ml, e.g., from 1 μg/ml to 100 μg/ml, e.g., from 2 μg/ml to 80 μg/ml, e.g., from 4 μg/ml to 40 μg/ml, e.g., from 5 μg/ml to 50 μg/ml, e.g., from 10 μg/ml to 50 μg/ml, e.g., from 15 μg/ml to 40 μg/ml, e.g., from 20 μg/ml to 30 μg/ml, e.g., 25μ/ml.

According to specific embodiments, the antioxidant is provided at a concentration range of 0.3-300 μg/ml, 3-300 μg/ml or 3-300 μg/ml in the composition e.g. hydrogel.

According to specific embodiments, the antioxidant is provided at a concentration range of 5-40 μg/ml in the composition e.g. hydrogel.

According to some embodiments of the invention, the antioxidant is provided at a concentration range of 0.3 mM (e.g. for Vitamin E 129 μg/ml) to 30 mM (e.g. for Vitamin E 12.9 mg/ml) in the hydrogel. For example, the antioxidant can be provided at a concentration range of 129 μg/ml-12.9 mg/ml, e.g., 200 μg/ml-10 mg/ml, e.g., 250 μg/ml-7.5 mg/ml, e.g., 300 μg/ml-5 mg/ml, 1 mg/ml-5 mg/ml, e.g., 1 mg/ml-4.5 mg/ml, e.g., 1 mg/ml-2.5 mg/ml.

According to specific embodiments, the antioxidant is provided at a concentration range of 0.001-100 mM. 0.001-50 mM, 0.01-100 mM, 1-100 mM, 1-50 mM in the composition e.g. hydrogel.

According to specific embodiments, the antioxidant is provided at a concentration range of 0.3-30 Mm, 0.6-30 mM, 1-30 mM, 1.5-30 mM, 2-30 mM, 2.5-30 mM, 3-30 mM, 5-30 mM, 10-30 mM, or 20-30 mM.

According to specific embodiments, the antioxidant is provided at a concentration range of 0.3-10 Mm, 0.3-5 mM, 0.3-3 mM, 0.6-10 mM, 0.6-5 mM, 0.6-3 mM, 1-10 mM, 1-5 mM or 1-3 mM.

According to specific embodiments, the antioxidant is provided at a concentration of 3 mM in the composition e.g. hydrogel.

According to specific embodiments, the ratio between the hyaluronic acid, the laminin polypeptide and the antioxidant (e.g. SOD) in the composition e.g. hydrogel is between HA 0.01 mg:laminin polypeptide 50 μg:antioxidant 250 μg per ml to HA 1.2 mg:laminin polypeptide 50 μg:antioxidant 250 μg per ml.

According to a specific embodiment, the ratio between the hyaluronic acid, the laminin polypeptide and the antioxidant (e.g. SOD) in the composition e.g. hydrogel is approximately HA 0.4 mg:laminin polypeptide 50 μg:antioxidant 250 μg per ml.

According to specific embodiments, the ratio between the hyaluronic acid, the laminin polypeptide and the antioxidant (e.g. Vitamin E) in the composition e.g. hydrogel is HA 0.1-1%:laminin polypeptide 0.1-100 μg/ml:antioxidant (e.g. Vitamin E) 0.1-10 mM.

According to a specific embodiment, the ratio between the hyaluronic acid, the laminin polypeptide and the antioxidant (e.g. Vitamin E) in the composition e.g. hydrogel is approximately HA 0.2%:laminin polypeptide 10 μg/ml:antioxidant (e.g. Vitamin E) 3 mM.

According to a specific embodiment, the ratio between the hyaluronic acid, the laminin polypeptide and the antioxidant in the composition e.g. hydrogel is approximately HA 0.4%:laminin polypeptide 10 μg/ml:antioxidant (e.g. Vitamin E) 3 mM.

According to a specific embodiment, the ratio between the hyaluronic acid, the laminin polypeptide and the antioxidant in the composition e.g. hydrogel is approximately HA 0.7%:laminin polypeptide 10 μg/ml:antioxidant (e.g. Vitamin E) 3 mM.

According to specific embodiments, the hyaluronic acid, the laminin polypeptide and the antioxidant are provided at a total concentration of about 0.01-0.6%.

According to specific embodiments, the hyaluronic acid, the laminin polypeptide and the antioxidant are provided at a total concentration of about 0.02-0.5%.

According to specific embodiments, the hyaluronic acid, the laminin polypeptide and the antioxidant are provided at a total concentration of about 0.2-0.4%.

According to specific embodiments, the hyaluronic acid, the laminin polypeptide and the antioxidant are provided at a total concentration of about 0.4%.

According to specific embodiments, the hyaluronic acid, the laminin polypeptide and the antioxidant are provided at a total concentration of about 0.2%.

According to specific embodiments, the hyaluronic acid, the laminin polypeptide and the antioxidant are provided at a total concentration of about 0.02%.

According to specific embodiments, the neuronal supporting agent and/or anti-gliotic agent is provided at a concentration of less than 5 μg/ml in the composition e.g. hydrogel.

According to specific embodiments, the neuronal supporting agent and/or anti-gliotic agent is provided at a concentration of 0.5-4.99 μg/ml, 0.5-4.5 μg/ml, 0.5-4 μg/ml, 0.5-3.5 μg/ml, 0.5-3 μg/ml, 0.5-2.5 μg/ml, 0.5-2 μg/ml or 0.5-1.5 μg/ml in the composition e.g. hydrogel.

According to specific embodiments, the neuronal supporting agent and/or anti-gliotic agent is provided at a concentration of 1-4.99 μg/ml, 1.5-4.99 μg/ml, 2-4.99 μg/ml, 2.5-4.99 μg/ml, 3-4.99 μg/ml, 3.5-4.99 μg/ml, 4-4.99 μg/ml or 4.5-4.99 μg/ml in the composition e.g. hydrogel.

According to specific embodiments, the neuronal supporting agent and/or anti-gliotic agent is provided at a concentration range of 5-300 μg/ml in the composition e.g. hydrogel.

According to specific embodiments, the neuronal supporting agent and/or anti-gliotic agent is provided at a concentration range of 10-300 μg/ml, 10-250 μg/ml, 10-200 μg/ml, 10-150 μg/ml, 10-100 μg/ml, 10-50 μg/ml, 10-40 μg/ml, 10-30 μg/ml or 10-20 μg/ml in the composition e.g. hydrogel.

According to specific embodiments, the neuronal supporting agent and/or anti-gliotic agent is provided at a concentration range of 1-500 μg/ml, 0.1-100 μg/ml, 0.1-50 μg/ml or 1-50 μg/ml in the composition e.g. hydrogel.

According to specific embodiments, the neuronal supporting and/or anti-gliotic agent is provided at a concentration of 10 μg/ml in the composition e.g. hydrogel.

According to specific embodiments of the invention, the hydrogel is lyophilized by methods well known in the art such that a dry matrix is obtained.

According to specific embodiments, the dry mix comprises less than 50%, less than 30%, less than 10%, less than 5%, less than 2%, less than 1%, or less than 0.5% water. It should be noted that water-free matrices can be preserved for long periods of time without being subjected to enzymatic degradation or contamination (e.g., by microorganisms).

Thus, according to specific embodiments, the composition described herein is formulated as a matrix.

As used herein the phrase “matrix” refers to a two-dimensional or a three-dimensional scaffold (also referred to herein as supporting framework) comprising the composition disclosed herein. The scaffold may further provide mechanical stability and support.

The matrix can be kept in a dry or wet form, or can be frozen according to the intended use.

According to specific embodiments, the dry matrix can be further hydrated in an aqueous solution (e.g., water) until a hydrogel is formed.

According to specific embodiments, the dimensions of the matrix vary according with the lesion (e.g. nerve injury e.g. spinal cord injury) to be treated. For example, the size of the matrix can be smaller than or substantially the same size as the lesion to be treated.

Alternatively, the size of the matrix can be larger than the lesion.

Scaffold material may comprise natural or synthetic organic polymers that can be gelled, or polymerized or solidified (e.g., by aggregation, coagulation, hydrophobic interactions, or cross-linking) into a two-dimensional or a three-dimensional structure.

The scaffold of some embodiments of the present invention may be made uniformly of a single polymer, co-polymer or blend thereof. However, it is also possible to form a scaffold according to the invention of a plurality of different polymers. There are no particular limitations to the number or arrangement of polymers used in forming the scaffold. Any combination which is biocompatible, may be formed into fibers, and degrades at a suitable rate, may be used.

Both the choice of polymer and the ratio of polymers in a co-polymer may be adjusted to optimize the stiffness of the scaffold. The molecular weight and cross-link density of the scaffold may also be regulated to control both the mechanical properties of the scaffold and the degradation rate (for degradable scaffolds). The mechanical properties may also be optimized to mimic those of the tissue at the implant site.

Polymers used in scaffold material compositions may be biocompatible, biodegradable and/or bioerodible and may act as adhesive substrates for cells. In exemplary embodiments, structural scaffold materials are easy to process into complex shapes and have a rigidity and mechanical strength suitable to maintain the desired shape under in vivo conditions.

In certain embodiments, the structural scaffold materials may be non-resorbing or non-biodegradable polymers or materials. Such non-resorbing scaffold materials may be used to fabricate materials which are designed for long term or permanent implantation into a host organism.

The phrase “non-biodegradable polymer”, as used herein, refers to a polymer or polymers which at least substantially (i.e. more than 50%) do not degrade or erode in vivo. The terms “non-biodegradable” and “non-resorbing” are equivalent and are used interchangeably herein. Examples of biocompatible non-biodegradable polymers which are useful as scaffold materials include, but are not limited to, polyethylenes, polyvinyl chlorides, polyamides such as nylons, polyesters, rayons, polypropylenes, polyacrylonitriles, acrylics, polyisoprenes, polybutadienes and polybutadiene-polyisoprene copolymers, neoprenes and nitrile rubbers, polyisobutylenes, olefinic rubbers such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates such as ethylene vinyl acetate copolymer, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers such as polytetrafluoroethylene and polyvinyl fluoride, polystyrenes, homopolymers and copolymers of styrene acrylonitrile, cellulose acetates, homopolymers and copolymers of acrylonitrile butadiene styrene, polymethylpentenes, polysulfones, polyesters, polyimides, polyisobutylenes, polymethylstyrenes, and other similar compounds known to those skilled in the art.

In other embodiments, the structural scaffold materials may be a “bioerodible” or “biodegradable” polymer or material.

The phrase “biodegradable polymer” as used herein, refers to a polymer or polymers which degrade in vivo, and wherein erosion of the polymer or polymers over time occurs concurrent with or subsequent to release of the composition. The terms “biodegradable” and “bioerodible” are equivalent and are used interchangeably herein.

Such bioerodible or biodegradable scaffold materials may be used to fabricate temporary structures. Examples of biocompatible biodegradable polymers which are useful as scaffold materials include, but are not limited to, polylactic acid, polyglycolic acid, polycaprolactone, and copolymers thereof, polyesters such as polyglycolides, polyanhydrides, polyacrylates, polyalkyl cyanoacrylates such as n-butyl cyanoacrylate and isopropyl cyanoacrylate, polyacrylamides, polyorthoesters, polyphosphazenes, polypeptides, polyurethanes, polystyrenes, polystyrene sulfonic acid, polystyrene carboxylic acid, polyalkylene oxides, alginates, agaroses, dextrins, dextrans, polyanhydrides, biopolymers such as collagens and elastin, alginates, chitosans, glycosaminoglycans, and mixtures of such polymers. In still other embodiments, a mixture of non-biodegradable and bioerodible and/or biodegradable scaffold materials may be used to form a biomimetic structure of which part is permanent and part is temporary.

According to specific embodiments, PLA, PGA and PLA/PGA copolymers are used for forming the scaffolds.

In an exemplary embodiment, scaffolds materials may comprise naturally occurring substances, such as, fibrinogen, fibrin, thrombin, chitosan, collagen, alginate, poly(N-isopropylacrylamide), albumin, collagen, synthetic polyamino acids, prolamines, polysaccharides such as alginate, heparin, and other naturally occurring biodegradable polymers of sugar units.

According to specific embodiments, the scaffolds are porous. The porosity may be controlled by a variety of techniques known to those skilled in the art.

According to a specific embodiment of the present invention, the scaffolds are fabricated from synthetic biomaterials and are capable of conducting electricity and naturally eroding inside the body. In an exemplary embodiment, the scaffolds comprise a biocompatible polymer capable of conducting electricity e.g. a polypyrrole polymer. Polyaniline, polyacetyline, poly-p-phenylene, poly-p-phenylene-vinylene, polythiophene, and hemosin are examples of other biocompatible polymers that are capable of conducting electricity and may be used in conjunction with the present invention. Other erodible, conducting polymers are well known (for example, see Zelikin et al., Erodible Conducting Polymers for Potential Biomedical Applications, Angew. Chem. Int. Ed. Engl., 2002, 41(1):141-144). Any of the foregoing electrical conducting polymers can be applied or coated onto a malleable or moldable scaffold.

The scaffolds may be made by any of a variety of techniques known to those skilled in the art. Salt-leaching, porogens, solid-liquid phase separation (sometimes termed freeze-drying), and phase inversion fabrication may all be used to produce porous scaffolds. Fiber pulling and weaving (see, e.g. Vacanti, et al., (1988) Journal of Pediatric Surgery, 23: 3-9) may be used to produce scaffolds having more aligned polymer threads. Those skilled in the art will recognize that standard polymer processing techniques may be exploited to create polymer scaffolds having a variety of porosities and microstructures.

Scaffold materials are readily available to one of ordinary skill in the art, usually in the form of a solution (suppliers are, for example, BDH, United Kingdom, and Pronova Biomedical Technology a.s. Norway). For a general overview of the selection and preparation of scaffolding materials, see the American National Standards Institute publication No. F2064-00 entitled Standard Guide for Characterization and Testing of Alginates as Starting Materials Intended for Use in Biomedical and Tissue Engineering Medical Products Applications”.

According to specific embodiments the scaffold comprises a biodegradable membrane e.g. a dura film such as Lyodura, AESCULAP.

According to specific embodiments, the composition, the matrix or the hydrogel of some embodiments of the present invention is comprised in an implantable tube. Such implantable tubes are well known in the art and disclosed for example in Jones et al. Int. J. Mol. Sci. 2016, 17, 1494 the contents of which are fully incorporated herein by reference and include, but are not limited to NeuraGen® nerve guide obtained from Integra, NeuroTube® from Synovis Micro, NeuroFlex™ and NeuroMatrix™ from Collagen Matrix, NeuroMend™ from Collagen Matrix, Neurolac® from Polyganics BV, Groningen, Netherlands, SaluTunnel™ from Salumedica LLC, Avance® from AxoGen, AxoGuard® from AxoGen, Nerbridge™ from Toyobo, NeuraWrap™ from Integra LifeSciences Co., and acellular nerve allograft.

According to specific embodiments of the invention, the composition, the hydrogel or the matrix further comprises ex-vivo seeded cells such as stem cells or differentiated cells (e.g. neuronal progenitor cells).

According to specific embodiments, the differentiated cells are neural cells.

According to specific embodiments, the cells are stem cells.

Non-limiting examples of stem cells which can be used by the invention include embryonic stem cells, induced pluripotent stem cells (iPS), neuronal progenitor cells, hematopoietic stem cells (e.g., bone marrow stem cells, cord blood cells, peripheral blood stem cells), adult stem cells and mesenchymal stem cells.

According to some embodiments of the invention, the stem cells are neuronal progenitor cells (such as those obtained from embryonic or fetal neuronal tissue or brain).

According to specific embodiments, the stem cells differentiate into neuronal cells when seeded in the composition, the matrix or the hydrogel.

Those of skills in the art are capable of determining when and how to implant the composition, the matrix or the hydrogel to thereby induce e.g. tissue formation within the subject. See for example, Artzi Z, et al., 2005, J. Clin. Periodontol. 32: 193-9; Butler C E and Prieto V G, 2004, Plast. Reconstr. Surg. 114: 464-73 and/or the Examples section which follows.

According to specific embodiments, the composition, the matrix or the hydrogel are implanted locally at or near the site of the injury.

Thus, according to specific embodiments, the composition, the matrix or the hydrogel is formulated for local administration.

For example, for treating spinal cord injuries, the composition, the matrix or the hydrogel is implanted directly into the lesion (e.g. into the epicenter of the injury), and near the lesion (e.g. at distance of approximately 0.5 cm from the injured site.). According to specific embodiments, the composition, the matrix or the hydrogel is injected into an implantable tube (e.g. NeuraGen® nerve guide obtained from Integra, NeuroTube® from Synovis Micro, NeuroFlex™ and NeuroMatrix™ from Collagen Matrix, NeuroMend™ from Collagen Matrix, Neurolac® from Polyganics BV, Groningen, Netherlands, SaluTunnel™ from Salumedica LLC, Avance® from AxoGen, AxoGuard® from AxoGen, Nerbridge™ from Toyobo, NeuraWrap™ from Integra LifeSciences Co.) which is implanted directly at or near the lesion. Following implantation the implants can be fixed by surgical adhesives (such as disclosed in e.g. Bhagat et al. Biomacromolecules 2017, 18, 3009-3039 e.g. gelatin-resorcinol-formaldehyde/glutaraldehyde glue (GRFG), cyanoacrylate glue, polysaccharide adhesive, polypeptide adhesive, polymeric adhesive, polyethylene glycol (PEG)-based hydrogel adhesive, biomimetic tissue adhesive, gecko-inspired tissue adhesive, sandcastle worm-inspired tissue adhesive, barnacle mimetic adhesive, caddisfly-inspired tissue adhesive, and fibrin glue e.g. Tisseel, BioGlue, CryoLife, Beriplast P, Hemaseel, Quixil, Bolheal, biocol, VIGuard F.S.); and finally the muscular and cutaneous planes are closed and sutured.

According to specific embodiments, the composition, the matrix or the hydrogel is implanted at least 1 week, at least 2 weeks, at least 3 weeks, at least 4 weeks, at least 2 months, at least 6 months, at least 12 months, at least 2 years following onset or diagnosis of the nerve injury, each possibility represents a separate embodiment of the present invention.

According to specific embodiments, treatment with the composition, the matrix or the hydrogel is effected at least 1 week, at least 2 weeks, at least 3 weeks, at least 4 weeks, at least 2 months, at least 6 months, at least 12 months, at least 2 years following onset or diagnosis of the nerve injury, each possibility represents a separate embodiment of the present invention.

According to specific embodiments, the composition, the matrix or the hydrogel is implanted at least 1 week following onset or diagnosis of the nerve injury.

According to specific embodiments, treatment with the composition, the matrix or the hydrogel is effected at least 1 week following onset or diagnosis of the nerve injury.

According to specific embodiments, the composition, the matrix or the hydrogel is implanted at least 2 weeks following onset or diagnosis of the nerve injury.

According to specific embodiments, treatment with the composition, the matrix or the hydrogel is effected at least 2 weeks following onset or diagnosis of the nerve injury.

According to specific embodiments, the composition, the matrix or the hydrogel is implanted within 5 years, within 4 years, within 3 years, within 2 years, within 1 year following onset or diagnosis of the nerve injury, each possibility represents a separate embodiment of the present invention.

According to specific embodiments, treatment with the composition, the matrix or the hydrogel is effected within 5 years, within 4 years, within 3 years, within 2 years, within 1 year following onset or diagnosis of the nerve injury, each possibility represents a separate embodiment of the present invention.

According to specific embodiments, the composition, the matrix or the hydrogel is implanted within 3 years following onset or diagnosis of said nerve injury.

According to specific embodiments, treatment with the composition, the matrix or the hydrogel is effected within 3 years following onset or diagnosis of said nerve injury.

According to specific embodiments, the composition, the matrix or the hydrogel is implanted 1 week-5 years, 1 week-3 years, 1 week-1 year, 2 weeks-5 years, 2 weeks-3 years, 1 week-1 year, 1 month-5 years, 1 month-3 years, 1 week-1 year following onset or diagnosis of the nerve injury, each possibility represents a separate embodiment of the present invention.

According to specific embodiments, treatment with the composition, the matrix or the hydrogel is effected 1 week-5 years, 1 week-3 years, 1 week-1 year, 2 weeks-5 years, 2 weeks-3 years, 1 week-1 year, 1 month-5 years, 1 month-3 years, 1 week-1 year following onset or diagnosis of the nerve injury, each possibility represents a separate embodiment of the present invention.

The components necessary to carry out any of the methods described herein (e.g. the composition) may, if desired, be presented in a pack or dispenser device, such as an FDA approved kit, or an article of manufacture (with packaging material), which may contain one or more unit dosage forms containing the active ingredient. The pack may, for example, comprise metal or plastic foil, such as a blister pack. The pack or dispenser device may be accompanied by instructions for administration, implantation and/or treating a subject. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. The compositions, matrix, hydrogel, biological sealant and/or implantable tube of some embodiments of the invention formulated in a compatible pharmaceutical carrier may also be prepared, placed in an appropriate container, and labeled for treatment of an indicated condition, as is further detailed above.

Further, the components necessary to carry out any of the methods described herein (e.g. the compositions, the matrix, the hydrogel, the biological sealant and/or the implantable tube), may be provided individually or may be comprised in a kit.

Thus, according to an aspect of the present invention, there is provided a kit comprising a composition comprising a hyaluronic acid, a laminin polypeptide and an antioxidant; and instructions for use in deferred treatment of a nerve injury in a subject in need thereof, wherein said deferred treatment is at least 1 week following onset or diagnosis of the nerve injury in the subject.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### The In-Vivo Effect of Grg and Agrg in a Rabbit Chronic Peripheral Nerve Injury Model

Materials and Methods:

Materials—All vendors and storage conditions are shown in Table 3 hereinbelow.

TABLE 3
list of materials

| | | | | | Storage |
|---|---|---|---|---|---|
| Materials | Name | Cat. No. | Manufacturer | Supplier | Conditions |
| Test Items | NeuraGen ® Nerve | NW340 | Interga | Tzamal | RT |
| | Guide | | | | |
| | Laminin peptide | 4107326 | Bachem | Bachem | −20° C. |
| | HA | HA15M-1 | Lifecore | Lifecore | −20° C. |
| | | | | Biomedical | |
| | DMSO | D5879 | Sigma | Sigma | RT |
| | Tocopherol | 613420 | Millipore | Millipore | 4° C. |
| | Copaxone | N/A | Teva | Medilink | 4° C. |
| Adhesion | TISSEEL Fibrin | N/A | Baxter | Baxter | 4° C. |
| material | Sealant | | | | |
| Antiseptic | Polydine solution | N/A | FLORIS | Vetmarket | RT |
| | | | ISRAEL | | |
| Antibiotic | Baytril | N/A | BAYER | Lidor | RT |
| | | | | chemicals | |
| Anesthesia | Ketamine | N/A | Vetqvinol | Vetmarket | RT |
| Items | Medetomidine | N/A | Eurovet | Vetmarket | RT |
| | Atipamezole | N/A | Eurovet | Vetmarket | RT |
| | hydrochloride | | | | |
| | Xylazine | N/A | SURUPET | Vetmarket | RT |
| | Isoflurane | N/A | Vetmarket | Vetmarket | RT |
| Analgesia | Buprenorphine | N/A | Vetmarket | Vetmarket | RT |
| Items | Fentanyl patch | N/A | Rafa | Vetmarket | RT |
| | Marcaine | N/A | Astrazenca | Vetmarket | RT |
| Euthanasia | Pentobarbital | N/A | CST | Vetmarket | RT |
| Item | Sodium | | | | |
| Histology | Formaldehyde 10% | 6450365F1 | BioLab | BioLab | RT |
| Item | | | | | |

Induction of peripheral nerve injury (Surgery 1)—The animals (female New Zealand rabbits) were anesthetized using intramuscular (IM) injection of 10% Ketamine (35 mg/kg) and 2% Xylazine (5 mg/kg). Following, the animals were placed on the surgery table and connected to an anesthetic machine that delivers a mixture of Isoflurane (1.5-3%) and oxygen at a rate of 0.5-15 liter/min. The area of the surgery was shaved, washed with ethanol and polyline solution; and covered with a sterile sheet to ensure sterile conditions. The operation on the tibial portion of the sciatic nerve was carried out on the left hind limb. The rabbit was in a prone position, the hind limbs abducted and the skin over the lateral and caudal aspect of the limb up to the lumbar midline was sheared. An incision of about 6 cm in length was made along the fusion line of the muscles. The fascia was sharply divided and the two muscles (biceps femoris and semimembranosus) were bluntly retracted to enable access to the sciatic, peroneal and tibial nerves. Using a microscope, the tibial portion of the sciatic nerve was exposed and separated from the peroneal portion. Following, the tibial portion of the sciatic nerve was transected proximally and distally removing 1 cm of length. The remaining removed nerve was discarded. The ends of the transected nerve were sutured to a muscle to prevent possible sprouting of axons. Following, the muscles were sutured using 3-0 vicryl threads and the skin was closed using special metal staples.

Treatment of the peripheral nerve injury (Surgery 2)—Nine weeks following induction of the injury (Surgery 1), the animals were re-anesthetized and the left hind limb was shaved, cleaned with soap and water followed by washing with ethanol and polydine solution. The operation was carried out by exposing the proximal and distal ends of the left tibial portion of the sciatic nerve and separating it from the muscles. The transected nerve ends were released and 3 mm from each of the transected end were removed. The proximal and distal ends of the nerve, 2.5 mm each, were fixed into 3 cm of NeuraGen® tube pre-immersed in saline, creating a 2.5 cm gap between the two ends, and micro-surgically reconnected using 10-0 epineurium sutures. Following, the treatment guiding regenerative gel (GRG or AGRG) was injected to the tube in a volume of 1 ml. The external connective area between the tube and the nerve is covered by Tisseel glue. As controls, an empty tube, or an autologous nerve graft was transplanted between the proximal and distal ends of the nerve (see Table 4 hereinbelow). Following, the muscles were sutured using 3-0 vicryl threads and the skin was closed using special metal staples. Subsequently, the animals were followed-up for period of 6 months.

TABLE 4
Rabbit chronic peripheral nerve injury experimental design

| Group No. | Group Size | Treatment |
|---|---|---|
| 1 | N = 11 | NeuraGen ® empty tube |
| 2 | N = 11 | NeuraGen ® + GRG (HA 0.4% + |
| | | Laminin10 μg/ml + |
| | | Tocopherol + DMSO 0.6%) |
| 3 | N = 11 | NeuraGen ® + AGRG (HA 0.4% + |
| | | Laminin10 μg/ml + Tocopherol + |
| | | DMSO 0.6% + Copaxone ® 10 μg /ml) |
| 4 | N = 8* | Autologous nerve graft |
| 5 | N = 8 | Untreated |

Clinical Observation and Incision Score—Animals were monitored for general clinical signs and scored for inflammatory parameters once weekly, following each surgery.

Body Weight Measurements—Animal body weight was measured on study day 0 prior to surgery and thereafter once weekly for the entire study period.

Compound Muscle Action Potentials (CMAPs)—Compound Muscle Action Potentials (CMAPs) were recorded prior to and following the surgical procedure in both hind limbs, using a Dantec™ KEYPONT® PORTABLE. During the measurements the animals were anesthetized using IP injection of a mixture of medetomidine (0.25 mg/Kg) and ketamine (15 mg/Kg). CMAPs were recorded by placing the stimulating needle electrodes at the sciatic notch and the recording needles at the gastrocnemius muscle. The ground electrode was placed on the thigh of the side of stimulation. The sciatic nerve was stimulated by a bipolar stimulating electrode with a pulse of 0.1 m/sec in duration. The stimulus intensity was increased gradually, until maximal amplitude CMAP was obtained. Latency and amplitude of the CMAPs were measured.

Immunohistochemistry for anti-MBP—At termination, the tibial portion of the sciatic nerve was harvested, grossly cut into 3 pieces—proximal, middle (injury site) and distal and followed by fixation in formalin 10%, processed and embedded in paraffin. Embedded tissues in paraffin blocks were sectioned at approximately 5 μM thickness, 2 slides per block, put on a glass slide and immunohistochemistry stained with Myelin Basic Protein (IHC:MBP, Zotal, Israel). The stained slides were subjected to histological evaluation and pictures acquisition was performed only on pathological changes and of representative rats. An area of interest (AOI) and spatial calibration were applied to each image. Following, RGB Histogram threshold was used to depict the brown stain and the Area and Area ratio (%) of each threshold were measured.

Statistical evaluation—Data is presented as means±SEM. In order to detect significant differences in the electrophysiological assessment, one-way ANOVA followed by Tukey's multiple comparisons or one-way ANOVA followed by Dunnett's multiple comparisons were applied (GraphPad). A p value <0.05 was considered to represent a significant difference. For the immunohistochemistry analyses one-tailed and two-tailed Student's T-test were applied. The P value for statistically significance was set to p<0.1, p<0.05 or p<0.01.

Animal care and use statement—The study was performed following approval of an application form submitted to the Committee for Ethical Conduct in the Care and Use of Laboratory Animals that stated that the study complied with the rules and regulations set forth.

Results:

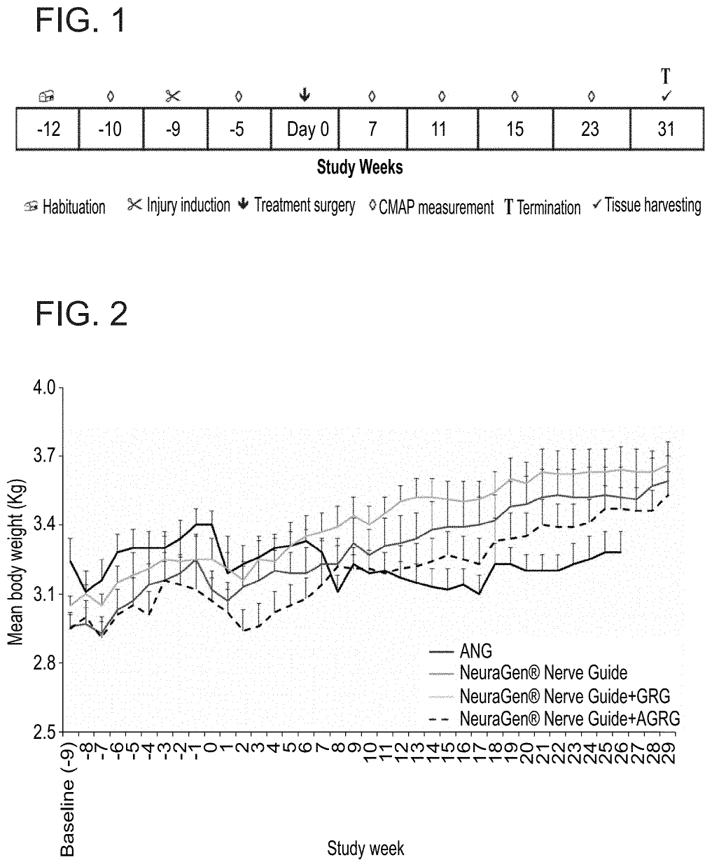

A schematic representation of the experimental procedure to evaluate the effect of GRG and AGRG in a rabbit chronic peripheral injury model is shown in FIG. 1. During the entire study period rabbits' body weight was determined. Following both surgeries the rabbit slightly lost weight, a phenomenon that is expected following such a procedure. During the following weeks, the animals recovered and gained weight, indicating the overall health of the animals was good (Table 5 hereinbelow and FIG. 2). Clinical observation indicated no redness or swelling of the area of the surgery in any of the operated rabbits. Although, in the autologous treatment a phenomena of severe heal pressure wounds and autotomy were observed in almost all rabbits, starting from week 17, which continued throughout the study. Therefore, in order to prevent such severe wounds, in the rest of the rabbits the operated limb was dressed and treated. This treatment helped to keep the wound small.

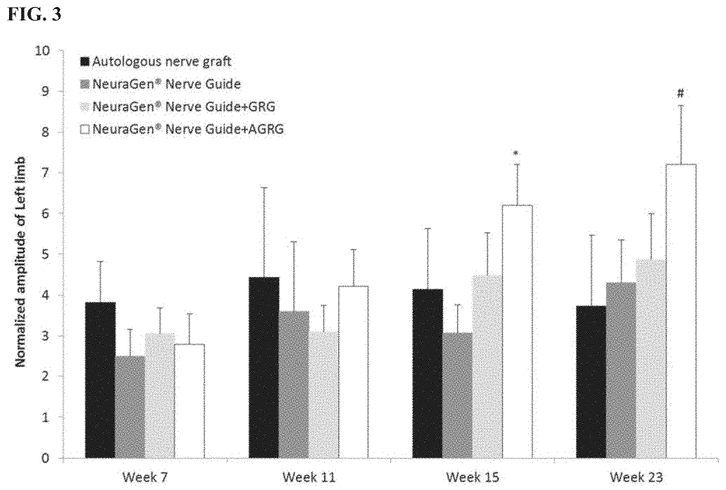

Following transection of the tibial portion of the sciatic nerve and preservation of the peroneal portion, electrophysiology assessments were determined by Compound Muscle Action Potential the (CMAPs) measured from the gastrocnemius muscle. The signal during the entire study in the injured limb (left) was markedly lower than that of the right limb throughout the study, with an exception of the autologous nerve graft treatment, in which the right hind limb was also injured. However, observing the normalized amplitude of the left limb to the amplitudes values measured between surgeries (week −5; FIG. 1 and Table 6 hereinbelow), the treatment with NeuraGen® Nerve Guide+AGRG showed a significant higher value at week 15 compared to that of the NeuraGen® Nerve Guide treatment (FIG. 3 and Table 6 hereinbelow; 6.20±1.01 vs. 3.08±0.69, respectively; p<0.05, using one-way ANOVA followed by Dunnett's test). This finding was also observed at week 23, when treatment with NeuraGen® Nerve Guide+AGRG showed the highest result in comparison to the other treatments. Further, treatment with NeuraGen® Nerve Guide+GRG also showed higher values than the NeuraGen® Nerve Guide treatment starting at week 15, indicating that the GRG also had a positive effect on nerve regeneration which was slower compared to the AGRG treatment. It is important to emphasize that the baseline values of the CMAPs, measured from both hind limbs, were within the normal range (right hind limb: 17.24±0.88 mV; left hind limb: 18.06±0.90 mV).

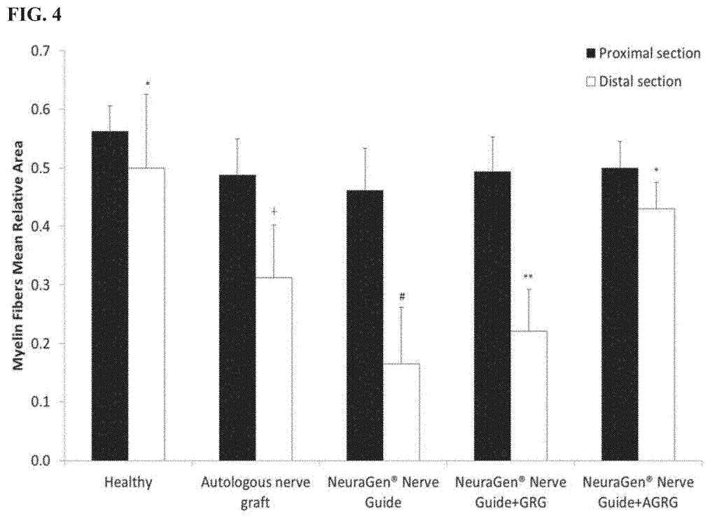

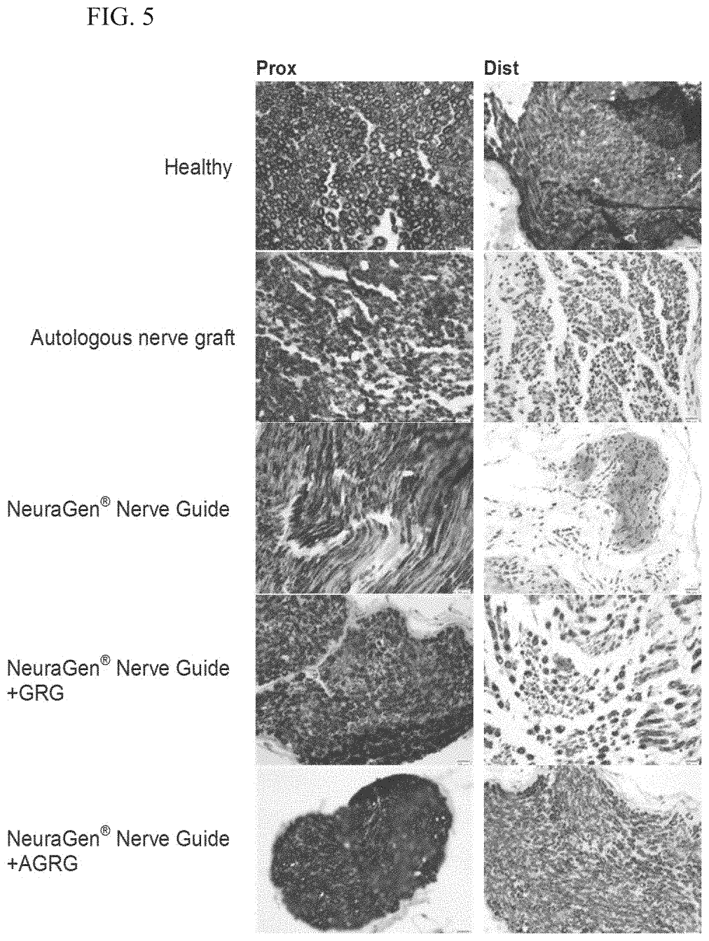

As week 31 post-treatment, the tibial portion of the sciatic nerve was harvested for histology and immunohistochemistry. H&E staining showed that the most proximal cross sections were unaffected or contained a mild vacuolization of the nerve's fibers and a very mild lymphocytic infiltration; and the distal sections were mostly mildly affected with fibers vacuolization. Following, to evaluate nerve reconstruction a myelin based protein (MBP) staining was performed. MBP staining of proximal sections demonstrated similar MBP mean relative area values for all treatments, which was also comparable to a healthy section (FIGS. 4-5 and Table 7 hereinbelow). However, upon examining the distal sections, a significant regeneration following treatment with GRG or AGRG was observed, compared to the NeuraGen® Nerve Guide treatment (FIGS. 4-5 and Table 7 hereinbelow; * p<0.1, two-tailed Student's T-test for the AGRG treatment).

Taken together, treatment with either GRG or AGRG in a model of a delayed peripheral nerve repair with massive nerve loss defect significantly improved and enhanced nerve regeneration, with AGRG showing even better results than the autologous nerve graft and close results to the healthy nerve at the time points tested.

TABLE 5
Mean body weight (Kg)

| | Study Weeks | | | | | | | | | | | |
|---|---|---|---|---|---|---|---|---|---|---|---|---|
| | MEAN | | | | | | SEM | | | | | |
| | Baseline | | | | | | Baseline | | | | | |
| Treatment | (−9) | −8 | −7 | −6 | −5 | −4 | (−9) | −8 | −7 | −6 | −5 | −4 |
| ANG* | 3.24 | 3.11 | 3.16 | 3.28 | 3.30 | 3.30 | 0.10 | 0.09 | 0.09 | 0.08 | 0.08 | 0.07 |
| NeuraGen ® Nerve | 2.96 | 2.97 | 2.93 | 3.03 | 3.07 | 3.14 | 0.06 | 0.07 | 0.07 | 0.09 | 0.11 | 0.09 |
| Guide | | | | | | | | | | | | |
| NeuraGen ® Nerve | 3.05 | 3.10 | 3.05 | 3.15 | 3.18 | 3.21 | 0.04 | 0.04 | 0.05 | 0.07 | 0.10 | 0.09 |
| Guide + GRG | | | | | | | | | | | | |
| NeuraGen ® Nerve | 2.95 | 3.00 | 2.91 | 3.01 | 3.05 | 3.01 | 0.06 | 0.07 | 0.07 | 0.11 | 0.12 | 0.10 |
| Guide + AGRG | | | | | | | | | | | | |

| | Study Weeks | | | | | | | | | | | |
|---|---|---|---|---|---|---|---|---|---|---|---|---|
| | MEAN | | | | | | SEM | | | | | |
| Treatment | −3 | −2 | −1 | 0 | 1 | 2 | −3 | −2 | −1 | 0 | 1 | 2 |
| ANG* | 3.30 | 3.34 | 3.40 | 3.40 | 3.19 | 3.23 | 0.07 | 0.08 | 0.07 | 0.06 | 0.16 | 0.08 |
| NeuraGen ® Nerve | 3.16 | 3.19 | 3.25 | 3.12 | 3.07 | 3.13 | 0.08 | 0.08 | 0.10 | 0.08 | 0.08 | 0.09 |
| Guide | | | | | | | | | | | | |
| NeuraGen ® Nerve | 3.25 | 3.24 | 3.25 | 3.25 | 3.21 | 3.16 | 0.10 | 0.10 | 0.11 | 0.09 | 0.07 | 0.08 |
| Guide + GRG | | | | | | | | | | | | |
| NeuraGen ® Nerve | 3.16 | 3.14 | 3.12 | 3.07 | 3.02 | 2.94 | 0.12 | 0.12 | 0.12 | 0.10 | 0.10 | 0.09 |
| Guide + AGRG | | | | | | | | | | | | |
| | Study Weeks | | | | | | | | | | | |
| | MEAN | | | | | | SEM | | | | | |
| Treatment | 3 | 4 | 5 | 6 | 7 | 8 | 3 | 4 | 5 | 6 | 7 | 8 |
| ANG* | 3.26 | 3.30 | 3.31 | 3.33 | 3.28 | 3.11 | 0.07 | 0.06 | 0.06 | 0.05 | 0.05 | 0.07 |
| NeuraGen ® Nerve | 3.16 | 3.20 | 3.19 | 3.19 | 3.23 | 3.23 | 0.10 | 0.09 | 0.09 | 0.11 | 0.11 | 0.11 |
| Guide | | | | | | | | | | | | |
| NeuraGen ® Nerve | 3.25 | 3.24 | 3.31 | 3.35 | 3.37 | 3.39 | 0.10 | 0.10 | 0.10 | 0.09 | 0.08 | 0.09 |
| Guide + GRG | | | | | | | | | | | | |
| NeuraGen ® Nerve | 2.96 | 3.02 | 3.05 | 3.08 | 3.14 | 3.22 | 0.10 | 0.09 | 0.09 | 0.09 | 0.09 | 0.09 |
| Guide + AGRG | | | | | | | | | | | | |
| | Study Weeks | | | | | | | | | | | |
| | MEAN | | | | | | SEM | | | | | |
| Treatment | 9 | 10 | 11 | 12 | 13 | 14 | 9 | 10 | 11 | 12 | 13 | 14 |
| ANG* | 3.23 | 3.19 | 3.20 | 3.17 | 3.15 | 3.13 | 0.04 | 0.08 | 0.08 | 0.07 | 0.08 | 0.08 |
| NeuraGen ® Nerve | 3.32 | 3.27 | 3.31 | 3.32 | 3.34 | 3.38 | 0.11 | 0.11 | 0.12 | 0.12 | 0.11 | 0.12 |
| Guide | | | | | | | | | | | | |
| NeuraGen ® Nerve | 3.44 | 3.40 | 3.45 | 3.50 | 3.52 | 3.52 | 0.08 | 0.08 | 0.07 | 0.07 | 0.08 | 0.08 |
| Guide + GRG | | | | | | | | | | | | |
| NeuraGen ® Nerve | 3.21 | 3.21 | 3.19 | 3.21 | 3.22 | 3.24 | 0.09 | 0.08 | 0.09 | 0.10 | 0.10 | 0.10 |
| Guide + AGRG | | | | | | | | | | | | |
| | Study Weeks | | | | | | | | | | | |
| | MEAN | | | | | | SEM | | | | | |
| Treatment | 15 | 16 | 17 | 18 | 19 | 20 | 15 | 16 | 17 | 18 | 19 | 20 |
| ANG | 3.12 | 3.14 | 3.10 | 3.23 | 3.23 | 3.20 | 0.09 | 0.07 | 0.08 | 0.09 | 0.07 | 0.07 |
| NeuraGen ® Nerve | 3.39 | 3.39 | 3.40 | 3.42 | 3.48 | 3.49 | 0.12 | 0.11 | 0.12 | 0.11 | 0.11 | 0.12 |
| Guide | | | | | | | | | | | | |
| NeuraGen ® Nerve | 3.51 | 3.50 | 3.51 | 3.54 | 3.60 | 3.58 | 0.08 | 0.09 | 0.08 | 0.09 | 0.09 | 0.09 |
| Guide + GRG | | | | | | | | | | | | |
| NeuraGen ® Nerve | 3.27 | 3.25 | 3.23 | 3.33 | 3.34 | 3.35 | 0.10 | 0.10 | 0.11 | 0.10 | 0.11 | 0.10 |
| Guide + AGRG | | | | | | | | | | | | |
| | Study Weeks | | | | | | | | | | | |
| | MEAN | | | | | | SEM | | | | | |
| Treatment | 21 | 22 | 23 | 24 | 25 | 26 | 21 | 22 | 23 | 24 | 25 | 26 |
| ANG* | 3.20 | 3.20 | 3.23 | 3.25 | 3.28 | 3.28 | 0.07 | 0.07 | 0.09 | 0.10 | 0.09 | 0.09 |
| NeuraGen ® Nerve | 3.52 | 3.53 | 3.52 | 3.52 | 3.53 | 3.52 | 0.12 | 0.11 | 0.12 | 0.13 | 0.12 | 0.13 |
| Guide | | | | | | | | | | | | |
| NeuraGen ® Nerve | 3.63 | 3.62 | 3.62 | 3.63 | 3.63 | 3.64 | 0.10 | 0.10 | 0.11 | 0.10 | 0.10 | 0.10 |
| Guide + GRG | | | | | | | | | | | | |
| NeuraGen ® Nerve | 3.40 | 3.39 | 3.39 | 3.41 | 3.47 | 3.47 | 0.11 | 0.10 | 0.10 | 0.10 | 0.10 | 0.09 |
| Guide + AGRG | | | | | | | | | | | | |

Study Weeks

| | MEAN | | | SEM | | |
|---|---|---|---|---|---|---|
| Treatment | 27 | 28 | 29 | 27 | 28 | 29 |
| ANG* | | | | | | |
| NeuraGen ® Nerve | 3.51 | 3.57 | 3.59 | 0.12 | 0.12 | 0.11 |
| Guide | | | | | | |
| NeuraGen ® Nerve | 3.63 | 3.63 | 3.66 | 0.10 | 0.09 | 0.10 |
| Guide + GRG | | | | | | |
| NeuraGen ® Nerve | 3.46 | 3.46 | 3.53 | 0.10 | 0.09 | 0.10 |
| Guide + AGRG | | | | | | |

*this group was conducted separately, as part of a developmental experiment, but the experiment design was the same as in the efficacy experiments. This group was terminated 3 weeks early than the other groups.

TABLE 6
CAMP normalized amplitude of the left hind limb

| | Study Weeks | | | | | | | |
|---|---|---|---|---|---|---|---|---|
| | MEAN | | | | SEM | | | |
| Treatment | 7 | 11 | 15 | 23 | 7 | 11 | 15 | 23 |
| ANG | 3.82 | 4.43 | 4.13 | 3.73 | 0.99 | 2.20 | 1.49 | 1.73 |
| NeuraGen ® | 2.48 | 3.60 | 3.08 | 4.31 | 0.67 | 1.69 | 0.69 | 1.04 |
| Nerve | | | | | | | | |
| Guide | | | | | | | | |
| NeuraGen ® | 3.05 | 3.10 | 4.48 | 4.87 | 0.64 | 0.64 | 1.04 | 1.12 |
| Nerve | | | | | | | | |
| Guide + | | | | | | | | |
| GRG | | | | | | | | |
| NeuraGen ® | 2.80 | 4.21 | 6.20* | 7.21# | 0.75 | 0.90 | 1.01 | 1.44 |
| Nerve | | | | | | | | |
| Guide + | | | | | | | | |
| AGRG | | | | | | | | |

The amplitude values of the CMAPs measured were normalized to the amplitude measured at 5 weeks prior the treatment surgery (surgery 2). Results are presented as mean ± SEM. Asterisk represents statistical significance:
*p < 0.05 vs. NeuraGen ® Nerve Guide, using one-way ANOVA followed by Dunnett's test;
#p < 0.05 vs. week 7, using one-way ANOVA followed by Tukey HSD test.

TABLE 7
Mean MBP per Area (%).

| | MEAN | | SEM | |
|---|---|---|---|---|
| Treatment | Prox | Dist | Prox | Dist |
| Healthy | 0.56 | 0.50* | 0.04 | 0.13 |
| ANG | 0.49 | 0.31^{†} | 0.06 | 0.09 |
| NeuraGen ® Nerve Guide | 0.46 | 0.17^{#} | 0.07 | 0.10 |
| NeuraGen ® Nerve Guide + | 0.49 | 0.22** | 0.06 | .07 |
| GRG | | | | |
| NeuraGen ® Nerve Guide + | 0.50 | 0.43* | 0.05 | 0.05 |
| AGRG | | | | |

The values of the MBP mean relative area are presented as mean ± SEM. Asterisk represents statistical significance:
*p < 0.1 using two-tailed Student's T-test vs. NeuraGen ® Nerve Guide;
**p < 0.01 using two-tailed Student's T-test vs. proximal section;
^{#}p < 0.05 using two-tailed Student's T-test vs. proximal section;
^{†}p < 0.1 using one-tailed Student's T-test vs. proximal section.

### Example 2

### The In-Vivo Effect of AGRG in a Rat Chronic Spinal Cord Injury Model

Materials and Methods

Materials—All vendors and storage conditions are shown in Table 8 hereinbelow.

TABLE 8
list of materials

| | | | | | Storage | |
|---|---|---|---|---|---|---|
| Materials | Name | Cat. No. | Manufacturer | Supplier | Conditions | |
| Spinal cord cover | NeuraWrap ™ | NW740 | Integra | Sponsor | RT | |
| | | NW540 | | | | |
| AGRG | Tocopherol | 613420 | Millipore | Millipore | 4° | C. |
| composition | Copaxone | N/A | Teva | MediPlus | 4° | C. |
| | Laminin peptide | 4107326 | Bachem | Bachem | −20° | C. |
| | HA | HA15M-1 | Lifecore | Lifecore | −20° | C. |
| | | | | Biomedical | | |
| | DMSO | D5879 | Sigma | Sigma | RT | |
| Adhesion material | TISSEEL Fibrin | NDC0338-4211-04 | Baxter | Baxter | 2-25° | C. |
| | Sealant | | | | | |
| Antibiotic | Baytril | N/A | Lidor | Vetmarket | RT | |
| | | | chemicals | | | |
| | Synthomycine | N/A | Rekah, Israel | Vetmarket | RT | |
| | (Chloramphenicol 5%) | | | | | |
| Antiseptic | Polydine solution | N/A | FLORIS | Vetmarket | RT | |
| | | | ISRAEL | | | |
| Anesthesia Items | Ketamine | N/A | Vetqvinol | Vetmarket | RT | |
| | Medetomidine | N/A | Eurovet | Vetmarket | RT | |
| | Atipamezole | N/A | Eurovet | Vetmarket | RT | |
| | hydrochloride | | | | | |
| Analgesia Items | Buprenorphine | N/A | Vetmarket | Vetmarket | RT | |
| | Dipyron | N/A | Ratiopharm | Vetmarket | RT | |
| | | | GmbH, Germany | | | |
| Euthanasia Item | Pentobarbital | N/A | CTS | Vetmarket | RT | |
| | Sodium | | | | | |
| Histology Item | Glutaraldehyde 2.5% | N/A | Electron | Bar Naor | 4°-(−18°) | C. |
| | | | Microscopy | | | |
| | | | Sciences | | | |

| | | | | |
|---|---|---|---|---|
| Calci-clear rapid | N/A | National | Bar Naor | RT |
| | | diagnostics | | |

Induction of spinal cord injury (Surgery 1)—The animals (Sprague Dawley rats) were anesthetized using intraperitoneal (IP) injection of ketamine (60 mg/kg) and medetomidine (0.25 mg/Kg). Following, the animals were placed on the surgery table and the area of the surgery was shaved, washed with ethanol and povidone iodine solution and covered with a sterile sheet to ensure sterile conditions. All surgical procedures were performed on anesthetized rats, using a high magnification neurosurgical microscope. The spinal cord was exposed via a dorsal approach. The overlying muscles were retracted, T7-T8 laminae removed, the spinal cord with dura was completely transected using micro-scissor and a 1 mm segment of the spinal cord was removed. The area of the laminectomy was covered by NeuraWrap™ Nerve Protector and its edges were glued using Tiseel glue to prevent leakage of the CSF. The muscular and cutaneous planes were closed and sutured.

Treatment of the spinal cord injury (Surgery 2)—One month following induction of the spinal cord injury (Surgery 1) the animals were re-anesthetized using IP injection of ketamine (60 mg/kg) and medetomidine (0.25 mg/K g). Following, the animals were placed on the surgery table, the area of the surgery was shaved, washed with ethanol and povidone iodine solution and covered with a sterile sheet to ensure sterile conditions. All surgical procedures were performed on anesthetized rats, using a high magnification neurosurgical microscope. The area of the spinal cord injury was exposed via a dorsal approach. The overlying muscles were retracted; and the T7-T8 area was exposed and widened. The entire area was cleaned from connective tissue and scar tissue, which allowed a clear gap of 2-3 mm between the proximal and distal ends of the spinal cord. Following, a 0.3 ml AGRG formulation was injected to the gap or left untreated (i.e. sham control) (Table 9 hereinbelow). A NeuraWrap™ Nerve Protector was used to cover the gap and glued using Tisseel glue to prevent leakage of the CSF or AGRG. The muscular and cutaneous planes were closed and sutured. The rats were followed-up for a period of 6 months following the 2^{nd} surgery.

TABLE 9
Rat chronic spinal cord injury experimental design

| Group No. | Group Size | Treatment |
|---|---|---|
| 1 | n = 16 | Untreated |
| 2 | n = 21 | AGRG |
| | | (HA 0.4% + Laminin 10 μg/ml + |
| | | Tocopherol + Copaxone ® 10 μg/ |
| | | ml + DMSO 0.6%) |

Body Weight Measurements—Animal body weight was measured on study day 0 prior to surgery and thereafter once weekly for the entire study period.

BBB locomotor rating score—The Basso, Beattie and Bresnahan Locomotor Rating Scale (BBB scale) was used to evaluate locomotion, graded from 0 (without performances) to 21 (with a complete-normal gait performance). The BBB scale is known as a valid and predictive measure of locomotor recovery able to distinguish behavioral outcomes due to different injuries and to predict anatomical alterations at the lesion center. Rats were monitored on week −1 (prior to the 2^{nd} surgery) and 0, 3, 7, 11, 15, 17, 19, 21 and 22/24 (following the 2^{nd} surgery) of the experiment.

Immunohistochemistry for anti-MBP—At termination, rats were perfused with saline+heparin, followed by fixation in glutaraldehyde (GA) 2.5%. Following 2 weeks of fixation, the tissues underwent decalcification, and the spinal cord was gently removed out of the vertebrae, and grossly cut into 3 pieces—cranial, middle (injury site) and caudal. Each piece was dehydrated and embedded in paraffin. Embedded tissues in paraffin blocks were sectioned at approximately 5 μM thickness, 2 slides per block, put on a glass slide and immunohistochemistry staining with Myelin Basic Protein (IHC:MBP, Zotal, Israel). The stained slides were subjected to histological evaluation and pictures acquisition was performed only on pathological changes and of representative rats. An area of interest (AOI) and spatial calibration were applied to each image. Following, RGB Histogram threshold was used to depict the brown stain and the Area and Area ratio (%) of each threshold were measured.

Statistical evaluation—The AGRG treatment was compared to the sham control untreated using Student's T-test (GraphPad). A p value <0.05 was considered to represent a significant difference.

Animal care and use statement—The study was performed following approval of an application form submitted to the Committee for Ethical Conduct in the Care and Use of Laboratory Animals that stated that the study complied with the rules and regulations set forth.

Results

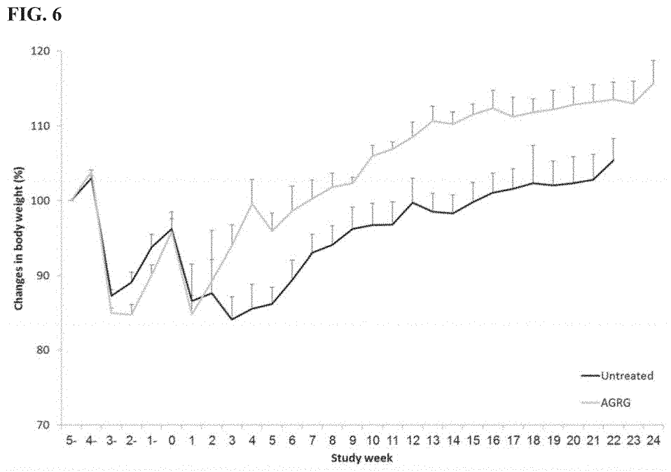

Rats were weighed once weekly during the entire study period. Following each surgery the rats lost weight, a phenomenon that is expected following such a procedure. During the following weeks, the rats recovered and gained weight back. From week 3 a significant difference between the AGRG treated group and the untreated group. Specifically, the AGRG treatment gained more weight compared to the baseline weight and to the untreated group (Tables 10-11 hereinbelow, FIG. 6).

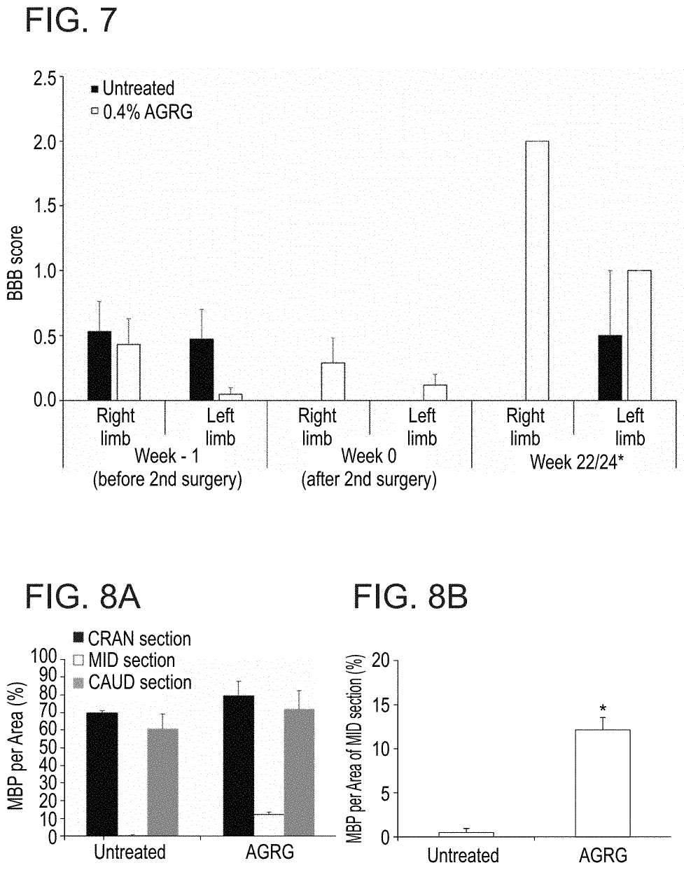

Further, rats treated with AGRG showed a higher mean BBB Locomotor score throughout the study, compared to the untreated group (FIG. 7, Table 12 hereinbelow). For example, on termination, i.e. week 22 for the untreated group or week 24 for the AGRG treatment, the mean BBB score of the right limb of the AGRG treated rats was 2.00±0.00 vs. 0.00±0.00 for the untreated rats. In addition, the mean BBB score of the left limb of the AGRG treated rats was 1.00±0.00 vs. 0.50±0.50 for the untreated rats on week 22/24.

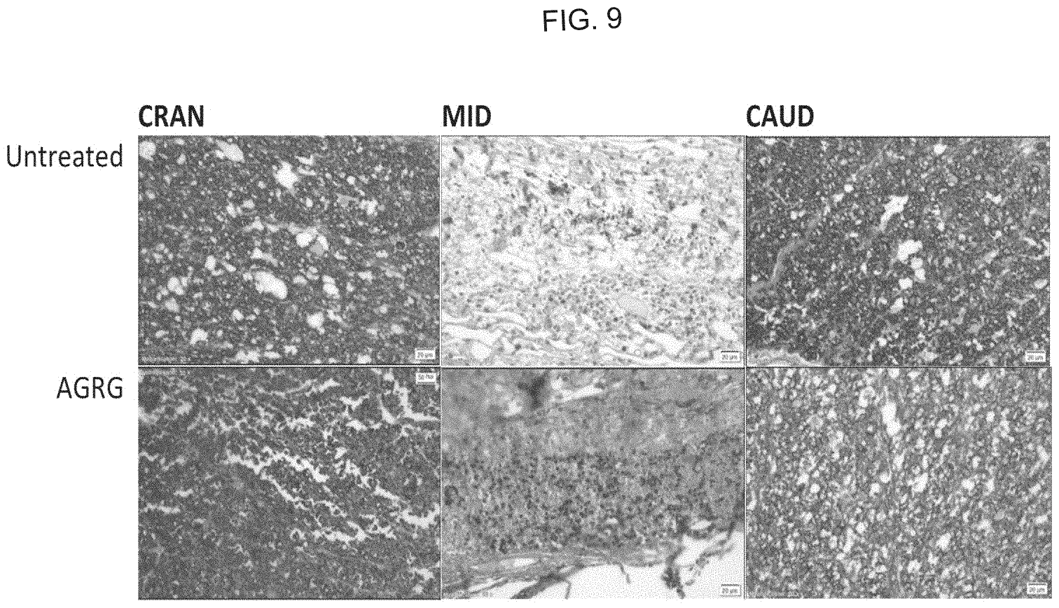

At weeks 22/24 post treatment, the spinal cords were harvested for histological and immunochemistry analysis. H&E staining showed a severe grade of tissue damage on the injury section (MID), while the cranial and caudal sections showed moderated tissue damage. Following, the samples were stained with MBP to evaluate nerve reconstruction. The MBP values of the MID sections following treatment with AGRG were significantly higher compared to the untreated group (FIGS. 8A-B and Table 13 hereinbelow; *p<0.01 using one-way ANOVA followed by Tukey's multiple comparisons). Cranial sections were used as individual control sections. In the caudal section only a minor loss of myelin was noted (FIGS. 8A and 9).

Taken together, treatment with AGRG in a model of a delayed spinal cord injury succeeded to regenerate the injured lesion after 6 months from treatment.

TABLE 10
Mean body weight (gr)

| | Study Weeks | | | | | |
|---|---|---|---|---|---|---|
| | | −4 | | | | 0 |
| | −5 | (1^{st} | | | | (2^{nd} |
| Treatment | (Baseline) | surgery) | −3 | −2 | −1 | surgery) |
| | MEAN | | | | | |
| Untreated | 339.25 | 349.56 | 296.19 | 302.25 | 318.07 | 326.00 |
| AGRG | 328.29 | 341.14 | 278.76 | 277.57 | 295.52 | 314.24 |
| | SEM | | | | | |
| Untreated | 2.99 | 3.44 | 4.12 | 5.44 | 5.59 | 6.81 |
| AGRG | 3.92 | 4.21 | 3.15 | 3.89 | 4.21 | 6.62 |

| | Study Weeks (after 2^{nd} surgery) | | | | | | | | | | | |
|---|---|---|---|---|---|---|---|---|---|---|---|---|
| Treatment | 1 | 2 | 3 | 4 | 5 | 6 | 7 | 8 | 9 | 10 | 11 | 12 |
| | MEAN | | | | | | | | | | | |
| Untreated | 292.00 | 289.25 | 282.33 | 287.33 | 289.33 | 300.33 | 312.33 | 315.67 | 323.00 | 324.67 | 325.00 | 334.67 |
| AGRG | 279.82 | 294.22 | 310.00 | 330.00 | 316.00 | 324.80 | 330.40 | 335.80 | 327.00 | 338.33 | 341.33 | 346.67 |
| | SEM | | | | | | | | | | | |
| Untreated | 13.90 | 23.61 | 10.97 | 11.89 | 8.69 | 9.84 | 9.06 | 9.24 | 10.44 | 10.65 | 10.79 | 10.84 |
| AGRG | 8.89 | 10.04 | 10.42 | 12.41 | 7.88 | 9.55 | 9.49 | 10.70 | 12.00 | 9.02 | 9.61 | 12.44 |
| | Study Weeks (after 2^{nd} surgery) | | | | | | | | | | | |
| Treatment | 13 | 14 | 15 | 16 | 17 | 18 | 19 | 20 | 21 | 22 | 23 | 24 |
| | MEAN | | | | | | | | | | | |
| Untreated | 330.67 | 330.00 | 335.00 | 339.33 | 341.00 | 345.00 | 344.00 | 345.00 | 346.50 | 354.00 | NA | NA |
| AGRG | 353.33 | 352.00 | 356.00 | 358.67 | 355.33 | 357.00 | 358.00 | 360.00 | 361.00 | 362.33 | 360.67 | 369.33 |
| | SEM | | | | | | | | | | | |
| Untreated | 8.69 | 8.66 | 9.64 | 9.61 | 9.85 | 18.00 | 12.00 | 13.00 | 12.50 | 11.00 | NA | NA |
| AGRG | 10.68 | 10.44 | 9.64 | 12.72 | 13.02 | 11.59 | 10.58 | 9.07 | 7.94 | 9.33 | 11.14 | 10.91 |

TABLE 11
Mean body weight changes in % (% from baseline, Week −5)

Study Weeks

| | | | | | | |
|---|---|---|---|---|---|---|
| | | −4 | | | | 0 |
| | −5 | (1^{st} | | | | (2^{nd} |
| Treatment | (Baseline) | surgery) | −3 | −2 | −1 | surgery) |

MEAN

| | | | | | | |
|---|---|---|---|---|---|---|
| Untreated | 100.00 | 103.03 | 87.28 | 89.09 | 93.79 | 96.22 |
| AGRG | 100.00 | 103.91 | 84.98 | 84.73 | 90.16 | 95.78 |

SEM

| | | | | | | |
|---|---|---|---|---|---|---|
| Untreated | 0.00 | 0.33 | 0.78 | 1.38 | 1.67 | 2.29 |
| AGRG | 0.00 | 0.22 | 0.67 | 1.36 | 1.29 | 1.81 |

| | Study Weeks (after 2^{nd} surgery) | | | | | | | | | | | |
|---|---|---|---|---|---|---|---|---|---|---|---|---|
| Treatment | 1 | 2 | 3 | 4 | 5 | 6 | 7 | 8 | 9 | 10 | 11 | 12 |

MEAN

| | | | | | | | | | | | | |
|---|---|---|---|---|---|---|---|---|---|---|---|---|
| Untreated | 86.61 | 87.63 | 84.10 | 85.58 | 86.18 | 89.46 | 93.04 | 94.04 | 96.22 | 96.71 | 96.81 | 99.71 |
| AGRG | 84.82 | 89.24 | 93.96 | 99.63 | 95.91 | 98.64 | 100.24 | 101.79 | 102.36 | 105.98 | 106.90 | 108.55 |

SEM

| | | | | | | | | | | | | |
|---|---|---|---|---|---|---|---|---|---|---|---|---|
| Untreated | 4.93 | 8.38 | 3.07 | 3.26 | 2.22 | 2.61 | 2.45 | 2.60 | 2.92 | 2.93 | 3.00 | 3.32 |
| AGRG | 2.51 | 2.88 | 2.80 | 3.20 | 2.38 | 3.31 | 2.52 | 1.91 | 0.77 | 1.40 | 0.96 | 2.00 |

| | Study Weeks (after 2^{nd} surgery) | | | | | | | | | | | |
|---|---|---|---|---|---|---|---|---|---|---|---|---|
| Treatment | 13 | 14 | 15 | 16 | 17 | 18 | 19 | 20 | 21 | 22 | 23 | 24 |
| | MEAN | | | | | | | | | | | |
| Untreated | 98.51 | 98.31 | 99.79 | 101.08 | 101.58 | 102.36 | 102.07 | 102.36 | 102.81 | 105.40 | NA | NA |
| AGRG | 110.67 | 110.25 | 111.51 | 112.32 | 111.27 | 111.80 | 112.16 | 112.81 | 113.14 | 113.53 | 113.01 | 115.73 |
| | SEM | | | | | | | | | | | |
| Untreated | 2.47 | 2.43 | 2.66 | 2.60 | 2.67 | 5.04 | 3.26 | 3.55 | 3.40 | 2.95 | NA | NA |
| AGRG | 1.91 | 1.62 | 1.39 | 2.44 | 2.54 | 1.79 | 2.56 | 2.34 | 2.35 | 2.30 | 2.95 | 3.02 |

TABLE 12
Mean (±SEM) Locomotor rating scale (BBB)

| | Weeks | | | | | | | | | | | |
|---|---|---|---|---|---|---|---|---|---|---|---|---|
| | Week −1 | | Week 0 | | | | | | | | | |
| | (before 2^{nd} | | (after 2^{nd} | | | | | | | | | |
| | surgery) | | surgery) | | 3 | | 7 | | 11 | | 15 | |
| | Right | Left | Right | Left | Right | Left | Right | Left | Right | Left | Right | Left |
| Treatment | Limb | Limb | Limb | Limb | Limb | Limb | Limb | Limb | Limb | Limb | Limb | Limb |
| | MEAN | | | | | | | | | | | |
| Untreated | 0.53 | 0.47 | 0.00 | 0.00 | 0.00 | 0.00 | 0.00 | 0.00 | 0.00 | 0.33 | 0.00 | 0.33 |
| AGRG | 0.43 | 0.05 | 0.29 | 0.12 | 0.63 | 0.88 | 0.20 | 1.20 | 1.33 | 0.67 | 1.33 | 1.00 |
| | SEM | | | | | | | | | | | |
| Untreated | 0.23 | 0.23 | 0.00 | 0.00 | 0.00 | 0.00 | 0.00 | 0.00 | 0.00 | 0.33 | 0.00 | 0.33 |
| AGRG | 0.20 | 0.05 | 0.19 | 0.08 | 0.32 | 0.40 | 0.20 | 0.97 | 0.88 | 0.33 | 0.88 | 0.00 |

| Weeks | | | |
|---|---|---|---|
| 17 | 19 | 21 | 22/24* |

| | | | | | | | | |
|---|---|---|---|---|---|---|---|---|
| | Right | Left | Right | Left | Right | Left | Right | Left |
| Treatment | Limb | Limb | Limb | Limb | Limb | Limb | Limb | Limb |

MEAN

| | | | | | | | | |
|---|---|---|---|---|---|---|---|---|
| Untreated | NA | NA | 0.00 | 0.50 | 0.00 | 0.50 | 0.00 | 0.50 |
| AGRG | 2.00 | 1.00 | 1.67 | 1.33 | 1.67 | 1.33 | 2.00 | 1.00 |

SEM

| | | | | | | | | |
|---|---|---|---|---|---|---|---|---|
| Untreated | NA | NA | 0.00 | 0.50 | 0.00 | 0.50 | 0.00 | 0.50 |
| AGRG | 0.58 | 0.00 | 0.33 | 0.33 | 0.33 | 0.33 | 0.00 | 0.00 |

**Week 22 and Week 24 were termination weeks for untreated and GRG treatments, respectively.

TABLE 13
Mean MBP per Area (%).

| | MEAN | | | SEM | | |
|---|---|---|---|---|---|---|
| Treatment | CRAN | MID | CAUD | CRAN | MID | CAUD |
| Untreated (n = 3) | 69.84 | 0.51 | 60.71 | 1.23 | 0.48 | 8.40 |
| AGRG (n = 2) | 79.42 | 12.14* | 71.76 | 8.07 | 1.39 | 10.39 |

The values of the MBP mean relative area are presented as mean ± SEM. Asterisk represents statistical significance:
*p < 0.01 using one-way ANOVA followed by Tukey's multiple comparisons vs. untreated.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method of deferred treatment of a nerve injury in a subject in need thereof, the method comprising implanting at least 1 week following onset or diagnosis of the nerve injury in the subject a composition comprising a hyaluronic acid, a laminin polypeptide and an antioxidant at or near the nerve injury of the subject, thereby treating the nerve injury in the subject.

2. The method of claim 1, wherein said implanting is effected at least two weeks following onset or diagnosis of said nerve injury.

3. The method of claim 1, wherein said implanting is effected within 3 years following onset or diagnosis of said nerve injury.

4-7. (canceled)

8. The method of claim 1, wherein said nerve injury is caused by a trauma and not by a disease.

9. The method of claim 1, wherein said nerve injury is a chronic nerve injury.

10. The method of claim 1, wherein said nerve injury is part of the peripheral nervous system (PNS).

11. The method of claim 1, wherein said nerve injury is part of the central nervous system (CNS).

12. The method of claim 11, wherein said nerve injury comprises spinal cord injury (SCI).

13. The method of claim 11, wherein said nerve injury comprises traumatic brain injuries (TBI) or traumatic optic neuropathy (TON).

14. The method of claim 1, wherein said antioxidant is vitamin E.

15. The method of claim 1, wherein said antioxidant is superoxide dismutase (SOD).

16. The method of claim 15, wherein said SOD comprises the amino acid sequence set forth by SEQ ID NO: 4.

17. The method of claim 1, wherein said laminin polypeptide is set forth by SEQ ID NO: 1.

18. The method of claim 1, wherein said antioxidant is vitamin E and said laminin polypeptide is set forth by SEQ ID NO: 1.

19. The method of claim 1, wherein said antioxidant is superoxide dismutase (SOD) comprising the amino acid sequence set forth by SEQ ID NO: 4 and said laminin polypeptide is set forth by SEQ ID NO: 1.

20-23. (canceled)

24. The method of claim 1, wherein said composition further comprises an anti-gliotic agent and/or a neuronal supporting agent.

25. (canceled)

26. The method of claim 24, wherein said antioxidant is vitamin E, said laminin polypeptide is set forth by SEQ ID NO: 1 and said agent is Copolymer 1.

27. The method of claim 24, wherein said antioxidant is superoxide dismutase (SOD) comprising the amino acid sequence set forth by SEQ ID NO: 4, said laminin polypeptide is set forth by SEQ ID NO: 1 and said agent is Copolymer 1.

28. (canceled)

29. The method of claim 1, wherein said composition is formulated as a hydrogel.

30. The method of claim 1, wherein said composition is formulated as a matrix.
